(19) United States
(12) Patent Application Publication
(10) Pub. No.: US 2002/0045574 A1
(43) Pub. Date: Apr. 18, 2002

(54) Glycopeptide antibacterial compounds and methods of using same

(76) Inventors: Ronald M. Kim, Hoboken, NJ (US); James Rocco Tata, Westfield, NJ (US); Kevin Chapman, Scotch Plains, NJ (US)

Correspondence Address: MCDERMOTT, WILL & EMERY, 600 13th Street, N.W., Washington, DC, 20005-3096, US

(21) Appl. No.: 09/840,927

(22) Filed: Apr. 25, 2001

Related U.S. Application Data
(60) Provisional application No. 60/199,359, filed on Apr. 25, 2000.

(51) Int. Cl. A61K 38/14; C07K 9/00

(52) U.S. Cl. 514/8; 530/322

(57) ABSTRACT

Vancomycin analogs in which the vancosamine residue is substituted on the vancosamine nitrogen with aryl substituents such as dichlorobenzyoxybenzyl, on the C₆ position with a polar substituent such as amino or substituted amino, and provided with functionality at the carboxyl such as amido derivatives, have improved activity against bacterial infection.

### CROSS REFERENCE TO RELATED APPLICATIONS

This application claims priority to Provisional Application Ser. No. 60/199,359, filed Apr. 25, 2000.

### FIELD OF THE INVENTION

The present invention relates to the preparation of derivatives of natural products and the determination of their activity. In particular, the present invention relates to novel derivatives of glycopeptide antibiotics, such as vancomycin, and their uses for the treatment of bacterial infection.

### BACKGROUND OF THE INVENTION

An example of a known glycopeptide antibiotic is vancomycin, which contains a disaccharide substituent linked to a heptapeptide structure. See Malabarba A., et al., Med. Res. Rev., 17(l):69-137 (1997a); Nagarajan R. et al., J. Chem. Soc. Chem. Comm. 1306-1307(1988); Nagaranjan R., Antimicr. Agents Chemother., 35:605-609 (1991); and Nagaranjan R., J. Antibiotics, 46:1181-1195 (1993). Vancomycin is effective against gram positive bacteria. However, vancomycin resistant strains have been recently observed, thus increasing the need for new and effective therapeutic agents.

The glycopeptides of the present invention are useful against many gram positive microorganisms, including vacomycin resistant enterococcus (VRE), methicillin resistant Staphylococcus aureus (MRSA), methicillin resistant Staphylococcus epidermidis (MRSE), and methicillin resistant coagulase negative Staphylococci (MR^{c}NS). The antibacterial compounds of the present invention thus comprise an important new contribution to the development of therapeutic regimens for treating infections caused by these difficult to control pathogens and resistant strains.

There is an increasing need for agents effective against such pathogens, which are at the same time relatively free from undesirable side effects. What is more, the physicochemical and pharmacological characteristics of candidate drugs, including their solubility, charge, hygroscopic characteristics, lipophilicity, bioavailability, tissue distribution, serum half-life and the like can play important roles in determining the success or failure of a candidate drug in the clinic.

It should be noted that antibiotics of the type that includes vancomycin are typically administered parenterally, that is intravenously. Hence, a relatively high clearance rate would not typically be a disadvantage, and as stated above, would be of potential great benefit to certain patients. Such intravenous formulations impose certain requirements on a drug, not the least of which is adequate solubility in the formulation medium. Thus, poorly soluble drugs may be unsuitable as a practical matter because the clinician is unable to dissolve the drug in a formulation, much less deliver adequate amounts of the drug via intravenous drip. Generally, the pH of the formulation is buffered to correspond to physiological pH, which is about 7.4. VA-dle some leeway is possible in the pH of an intravenous formulation, pain at the site of injection typically limits the useful range of pH to no less than about 5 to no greater than about 8. Preferably, the pH of an intravenous formulation ranges from about 6-8, more preferably from about 7-8 and most preferably at or about physiological pH (e.g., about 7.2-7.6).

Hence, there has been an on-going search for compounds that exhibit not only increased potency against resistant strains but also the physicochemical and pharmacological characteristics that enhance the effectiveness of a candidate compound and which may determine ultimately its acceptance in the clinic and resulting commercial success.

### SUMMARY OF THE INVENTION

The present invention provides new analogs of vancomycin, which exhibit enhanced biological activity and improved physicochemical and pharmacological characteristics. The overall properties of these analogs inform of their potential as drug candidates for treating infections caused by certain pathogens, including various strains of drug resistant bacteria. Accordingly, a general method is provided for the preparation of such compounds, along with methods of using them for the treatment of vertebrate conditions, including those afflicting mammals and especially those suffered by humans. Such conditions typically, although not exclusively, involve infections and other pathological conditions caused by bacteria and other microorganisms.

In particular, it has been observed that certain substituents positioned at the amine nitrogen of vancosamine and at the C-6 position of the glucose of vancomycin alternatively with other substitution, provide enhanced biological activity and give rise to desirable physicochemical and pharmacological characteristics, all of which improve a candidate drug's chances of success beyond the lab bench and in the clinic. More particularly, the present invention provides for certain substituents on the amine nitrogen of vancosamine, combined with substitution of a polar group at the C₆ position.

A wide range of possible polar substituents can be positioned at the glucose C-6 position of vancomycin. Polar substituents are substituents that bear a charge or possess the capacity to bear a charge, either positive or negative, at some useful range of pH, but preferably at or about physiological pH, enhance biological activity and/or provide advantageous physicochemical and/or pharmacological characteristics. Most preferably, the polar substituent is part of an N-substituent (that is, an amine or amine based substituent) at the C-6 position, including but not limited to a free amine, substituted amines, alpha-amino acid amides, carboxylic acid amides (e.g., the carboxylic acid amide obtained from the reaction of a C-6 amine with for example succinic acid, other diacids, anhydrides, or other bifunctional acids), quaternary ammonium salts and the like.

The compounds of the invention, including their pharmaceutically acceptable salts, are represented by the general Formula, presented below:

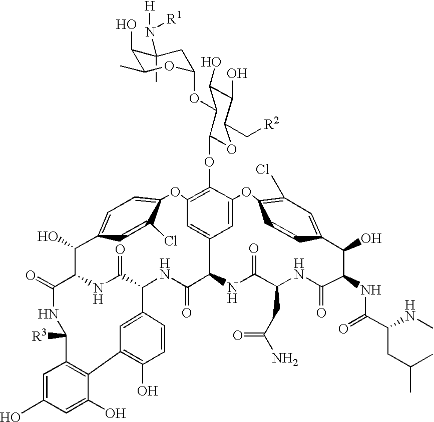

R¹ is XR^{a}; wherein X is absent or XR^{a} is

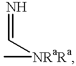

—SO₂R^{a}, —SO₂N^{a}R^{a}, —COOR^{a}, —CONR^{a}R^{a}, —COR^{A} when R^{a} is not hydrogen;

Each R^{a} is independently hydrogen, alkyl, aryl, heteroaryl, substituted alkyl, substituted aryl, substituted heteroaryl; wherein

(i) each of the substituents on substituted alkyl is independently

(a) halogen,

(b) cyano,

(c) OR^{b}

(d) NR^{b}R^{c}

(e) COOR^{b}

(f) CONR^{b}R^{c},

(g) SR^{b}

(h) —SO₂R^{b}

(i) SO₂NR^{b}R^{b}

(j) aryl,

(k) aryl substituted with one or more substituents independently selected from halogen, cyano, OR^{b}, SR^{b}, COOR^{b}, CONR^{b}R^{c}, NR^{b}R^{c}, SO₂R^{b}, SO₂NR^{b}R^{c}, alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};

(l) heterocycle, or

(m) heteroaryl substituted with one or more substituents independently selected from halogen, cyano, OR^{b}, SR^{b}, COOR^{b}, CONR^{b}R^{c}, NR^{b}R^{c}, SO₂R^{b}, SO₂NR^{b}R^{c}, alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};

(ii) each of the substituents on substituted aryl is independently

(a) halogen,

(b) cyano,

(C) OR^{b}

(d) NR^{b}R^{c}

(e) COOR^{b}

(f) CONR^{b}R^{c},

(g) SR^{b}

(h) SO₂R^{b}

(i) SO₂NR^{b}R^{b}

(j) aryl,

(k) aryl substituted with one or more substituents independently selected from halogen, cyano, OR^{b}, SR^{b} , COOR^{b}CONR^{b}R^{c}, NR^{b}R^{c}, SO₂R^{b}, SO₂R^{b}R^{c} alkyl, alkyl substituted with R^{b} , fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};

(l) heterocycle, or

(m) heteroaryl substituted with one or more substituents independently selected from halogen, cyano, OR^{b}, SR^{b} , COOR^{b}CONR^{b}R^{c}, NR^{b}R^{c}, SO₂R^{b}, SO₂NR^{b}R^{c} alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};

(n) alkyl,

(o) alkyl substituted with R^{b};

(p) alkenyl,

(q) alkenyl substituted with R^{b};

(r) alkynyl,

(s) alkynyl substituted with R^{b};

(iii) each of the substituents on substituted heteroaryl is independently

(a) halogen,

(b) cyano,

(C) OR^{b}

(d) NR^{b}R^{c}

(e) COOR^{b}

(f) CONR^{b}R^{c},

(g) SR^{b}

(h) SO₂R^{b}

(j) aryl,

(k) aryl substituted with one or more substituents independently selected from halogen, cyano, OR^{b}, SR^{b}, COOR^{b}CONR^{b}R^{c}, NR^{b}R^{c}, SO₂R^{b}, SO₂NR^{b}R^{c}, alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, heteroaryl substituted with R^{b};

(l) heterocycle, or

(m) heteroaryl substituted with one or more substituents independently selected from halogen, cyano, OR^{b}, SR^{b}, COOR^{b} CONR^{b}R^{c}, NR^{b}R^{c}, SO₂R^{b}, SO₂NR^{b}R^{c} alkyl, alkyl substituted with R^{b} , fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};

(n) alkyl,

(o) alkyl substituted with R^{b};

(p) alkenyl,

(q) alkenyl substituted with R^{b};

(r) alkynyl,

(s) alkynyl substituted with R^{b};

or R^{a} and R^{a} together with the nitrogen to which they are attached form C₃-C₆ heterocycloalkyl consisting of from 2 to 5 carbons atoms and froml or 2 nitrogen, oxygen, and sulfur atoms. The heterocycloalkyl may be substituted with alkyl, aryl, heteroaryl, OR^{b}, NR^{c}R^{b}, COOR^{b}, CONR^{b}R^{c}, substituted alkyl, substituted aryl, or substituted heteroaryl as defined above;

R^{b} and R^{c} are each independently hydrogen, alkyl, aryl, heteroaryl, substituted alkyl substituted with 1 to 3 groups of R^{x}, substituted aryl substituted with 1 to 3 groups of R^{y}, or substituted heteroaryl substituted with 1 to 3 groups of R^{z}; wherein

(i) wherein Rx represents:

(a) halogen,

(b) cyano,

(c) OH, O-alkyl

(d) N(alkyl)₂, NH-alkyl

(e) COOH, COO-alkyl

(f) CON(alkyl)₂, CONH-alkyl,

(g) SO₂N(alkyl)₂, SO₂NH-alkyl,

(h) aryl,

(i) aryl substituted with one or more substituents independently selected from halogen, cyano, hydroxy, O-alkyl, alkyl, fluorinated alkyl, COOH, COO-alkyl, CONH-alkyl, CON(alkyl)₂, and N(alkyl)₂;

(j) heterocycle, or

(k) heterocycle substituted with one or more substituents independently selected from from halogen, cyano, hydroxy, O-alkyl, alkyl, fluorinated alkyl, COOH, COO-alkyl, CONH-alkyl, CON(alkyl)₂, and N(alkyl)₂;

(ii) R^{y} represents:

(a) halogen,

(b) cyano,

(c) OH, O-alkyl

(d) N(alkyl)₂, NH-alkyl

(e) COOH, COO-alkyl

(f) CON(alkyl)₂, CONH-alkyl,

(g) SO₂N(alkyl)₂, SO₂NH-alkyl

(h) aryl,

(i) aryl substituted with one or more substituents independently selected from halogen, cyano, hydroxy, O-alkyl, alkyl, fluorinated alkyl, COOH, COO-alkyl, CONH-alkyl, CON(alkyl)₂, and N(alkyl)₂;

(j) heterocycle, or

(k) heterocycle substituted with one or more substituents independently selected from from halogen, cyano, hydroxy, O-alkyl, alkyl, fluorinated alkyl, COOH, COO-alkyl, CONH-alkyl, CON(alkyl)₂, and N(alkyl)₂;

(iii) where RZ represents:

(a) halogen,

(b) cyano,

(c) OH, O-alkyl

(d) N(alkyl)₂, NH-alkyl

(e) COOH, COO-alkyl

(f) CON(alkyl)₂, CONH-alkyl,

(g) SO₂N(alkyl)₂, SO₂NH-alkyl

(h) aryl,

(i) aryl substituted with one or more substituents independently selected from halogen, cyano, hydroxy, O-alkyl, alkyl, fluorinated alkyl, COOH, COO-alkyl, CONH-alkyl, CON(alkyl)₂, and N(alkyl)₂;

(j) heterocycle, or

(k) heterocycle substituted with one or more substituents independently selected from from halogen, cyano, hydroxy, O-alkyl, alkyl, fluorinated alkyl, COOH, COO-alkyl, CONH-alkyl, CON(alkyl)₂, and N(alkyl)₂;

or R^{b} and R^{c} together with the nitrogen to which they are attached form C₃-C₆ heterocycloalkyl consisting of from 2 to 5 carbons atoms and from 1 to 2 nitrogen, oxygen, and sulfur atoms. The heterocycloalkyl may be substituted with alkyl, aryl, heteroaryl, O-alkyl, NHalkyl, COOH, COO-alkyl, CONH-alkyl, CON(alkyl)₂, SO₂N(alkyl)₂, SO₂NH-alkyl, substituted alkyl, substituted aryl, or substituted heteroaryl as previously defined in R^{x}, R^{y} and R^{z}, respectively, above;

R² is NR^{a}R^{a}, SR^{a},

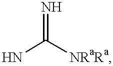

and OR^{a} wherein OR^{a} is not OH;

R³ is CONR^{a}R^{a} or COOR^{a} wherein COOR^{a} is not COOH and R^{a} does not contain as a sub-structure a generally recognized antibacterial agent.

Preferred compounds are those wherein R¹ is unsubstituted or substituted benzyloxybenzyl, preferably substituted with one or more halogens such as chlorine or unsubstituted or substituted biphenylbenzyl; R² is HN₂, or substituted amino such as hydroxy alkylamino, phenylalkylenamino, or a heterocyclic group such as morpholino or piperidino and R³ is morpholinylamido, hydroxyallsoxyalkoxylalkyleneamido, aminoalkyleneamido, or azidoallsoxyalkoxyallsoxyallsyleneamido.

The invention is also directed to pharmaceutical compositions, including enteral and parenteral formulations of the compounds disclosed herein. Also disclosed are methods of determining the biological activities of the various compounds of interest to the invention, as well as those of lesser interest. Methods of preparing the compounds and of utilizing same in a treatment regimen are also described and contemplated.

A more detailed description of the preferred embodiments of the invention follows for the benefit of the reader. Additional objects of the invention will become apparent to the reader after consideration of the entire disclosure.

### DETAILED DESCRIPTION OF THE INVENTION

As stated above, it has been observed that the vancomycin analogs of this invention possess enhanced biological activity relative to those conventional substituents that do not fall within the scope of the invention. These vancomycin analogs consistently exhibit an increase in activity over those vancomycin analogs bearing conventional substituents.

The term “alkyl” refers to a monovalent alkane (hydrocarbon) derived radical comprising 1 to about 20 carbon atoms connected by single or multiple bonds, unless otherwise indicated. The alkyl group may be straight, branched, or cyclic. Examples of alkyl groups include, but are not limited to, methyl, ethyl, propyl, isopropyl, butyl, secbutyl, t-butyl, pentyl, cyclopentyl, hexyl and cyclohexyl. The term “alkylene” refers to a hydrocarbon radical comprising 1 to about 20 carbon atoms connected by single or multiple bonds, unless otherwise indicated, and which is bound to other functional or chemical groups of the molecule at least at two sites. Examples of alkylene groups include, but are not limited to, —CH₂—, —CH₂CH₂— —CH₂CH₂CH₂— —CH₂ (CH₃)CH₂—, and the like, wherein each dash represents a point of attachment to another chemical or functional group of the molecule. When substituted, alkyl and alkylene groups may be substituted with substituent groups at any available point of attachment. When an alkyl group is described as being substituted with an alkyl group, such a phrase is used interchangeably with “branched alkyl group.”

The term “cycloalkyl” is a species of alkyl and is a group comprising about 3 to about 15 carbon atoms, without alternating or resonating double bonds between carbon atoms. It may also contain from 1 to 4 fused rings.

The term “aryl” refers to a group derived from a non-heterocyclic aromatic group having from six to about twenty carbon atoms and from one to four rings, which may be fused, connected by single bonds, or both. An aryl group may be substituted by one or more of alkyl, aralkyl, heterocyclic, heterocyclicalkyl, heterocycliccarbonyl, halo, hydroxyl, protected hydroxyl, amino, nitro, cyano, alkoxy, aryloxy, aralkyloxy, aroyloxy, alkylamino, dialkylamino, trialkylammonium, alkylthio, alkanoyl, alkanoyloxy, alkanoylamido, alkylsulfonyl, arylsulfonyl, aroyl, aralkanoyl, alkyloxycarbonyl, aralkyloxycarbonyl, aminocarbonyl, alkylaminocarbonyl, dialkylaminocarbonyl and the like.

The term “aralkyl” refers to an alkyl group bearing an aryl group substituent.

The terms “heterocyclic”, “heterocycle”, or “heteroaryl” refers to a cyclic hydrocarbon group in which at least one of the ring positions is occupied by a heteroatom. A heterocyclic compound may have from one to about four rings, which may be fused, connected by single bonds, or both. A heterocyclic group may comprise from three to about twenty ring atoms, which atoms may be chosen from carbon, nitrogen, oxygen, or sulfur as long as at least one heteroatom is present. A heterocyclic group may have up to 1, 2, or 3 double bonds per ring, thus allowing for an aromatic system. A heterocyclic group may be substituted by one or more of alkyl, aryl, aralkyl, halo, hydroxyl, protected hydroxyl, amino, nitro, cyano, alkoxy, aryloxy, aralkyloxy, aroyloxy, alkylamino, dialkylamino, trialkylammonium, alkylthio, alkanoyl, alkanoyloxy, alkanoylamido, alkylsulfonyl, arylsulfonyl, aroyl, aralkanoyl, alkyloxycarbonyl, aralkyloxycarbonyl, aminocarbonyl, alkylaminocarbonyl, dialkylaminocarbonyl and the like.

The term “alkenyl” refers to a monovalent alkene group comprising up to about 20 carbon atoms which contains at least one double bond between carbon atoms. The alkene group may be straight chained or branch chained. Examples include vinyl, propenyl, butenyl and pentenyl groups.

The term “alkynyl” refers to a monovalent alkyne group comrpsing up to about 20 carbon atoms which contains at least one triple bond between carbon atoms. The alkyne group may be straight or branch chained. Examples are propynyl, butynyl and pentynyl.

A generally recognized antibacterial agent referred to above as a sub-structure means any antibacterial agent whose structure is known in the art and is defined in the Merck Index.

The term “heteroatom” means an atom other than carbon or hydrogen, but is generally associated with the atoms N, 0, or S, selected on an independent basis.

The term “halogen” or “halo” refer to fluorine, chlorine, bromine, or iodine.

The terms “alkoxy,” “aryloxy” and “aralkyloxy” refer to a chemical group in which an oxygen atom is covalently bound to an alkyl, aryl, or aralkyl group, respectively.

The terms “alkanoyl,” “aroyl” and “aralkanoyl” refer to chemical groups in which a carbonyl group is covalently bound to an alkyl, aryl, or aralkyl group, respectively.

The term “heterocyclicalkyl” or “heterocycliccarbonyl” refers to chemical groups in which a heterocyclic group is covalently bound to an alkyl or carbonyl group, respectively.

When a group is termed “substituted,” unless otherwise indicated, this means that the group contains from 1 to the number of substituents which can be substituted on the group.

When a functional group is termed “protected,” this means that the group is in a temporary, modified form to inhibit the participation of the protected site in a particular reaction sequence intended to effect some change elsewhere in the molecule. Suitable protecting groups for the compounds of the present invention will be recognized from the present application taking into account the level of skill in the art, and with reference to standard textbooks, such as Greene, T. W. et al. “Protective Groups in Organic Synthesis” Wiley, New York (1991). In addition, examples of suitable protecting groups are presented throughout the specification.

In some of the glycopeptide compounds of the present invention a hydroxyl-protect group might be required. Such conventional protecting groups consist of known groups, which are used to protectively block the hydroxyl group during the synthetic procedures described herein. These conventional blocking groups are readily removable; i.e., they can be removed, if desired, by procedures that will not cause cleavage or other disruption of the remaining portions of the molecule. Such procedures include chemical and enzymatic hydrolysis, treatment with chemical reducing or oxidizing agents under mild conditions, treatment with a transition metal catalyst, a nucleophile and catalytic hydrogenation.

Examples of suitable C-6 hydroxyl protecting groups include, but are not limited to, triethylsilyl, t-butyldimethylsilyl, o-nitrobenzyloxycarbonyl, p-nitrobenzyloxy carbonyl, benzyloxycarbonyl, allyloxycarbonyl, t-butyloxycarbonyl, 2,2,2-trichloro ethyloxycarbonyl and the like.

The glycopeptide compounds of the present invention are useful per se and in their pharmaceutically acceptable salt and ester forms for the treatment of bacterial infections in animal and human subjects. The term “pharmaceutically acceptable ester, salt, or hydrate” refers to those esters, salts, or hydrated forms of the compounds of the present invention, which would be apparent to the medicinal chemist. Such forms include, but are not limited to, those that are substantially non-toxic and which may favorably affect the pharmacokinetic properties of said compounds, such as palatability, absorption, distribution, metabolism and excretion. Other factors, more practical in nature, which are also important in the selection, are cost of the raw materials, ease of crystallization, yield, stability, solubility, hygroscopicity and flowability of the resulting bulk drug. Conveniently, pharmaceutical compositions may be prepared from the active ingredients in combination with pharmaceutically acceptable carriers. Thus, the present invention is also concerned with pharmaceutical compositions and methods of treating bacterial infections utilizing as an active ingredient the novel glycopeptide compounds of the present invention, particularly the vancomycin-like glycopeptide compounds disclosed herein.

The pharmaceutically acceptable salts referred to above also include acid addition salts. Thus, the Formula I compounds can be used in the form of salts derived from inorganic or organic acids. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, undecanoate and the like.

Protection of amines by a similar group requires using excess acylation reagent while selective protection of the N-methyl leucine residue is known, allowing selective functionalization of the vancosamine amine group. See, Pavlov et al., J. Antiobiotics, 1993, 46, 1731. Selectively, introducing the mesitylenesulfonyl group at the glucose-6-position differentiates this position from the other hydroxyl groups and allows further reaction to displace the mesitylenesulfonyl group, affording many derivatives. A variety of functional groups are introduced at the glucose-6 position by using common methods for nucleophilic displacement of primary arylsulfonyl groups directly, or by further synthetic modification of initial displacement products, including azido and iodo groups. For example, the iodo group is displaced by a variety of nucleophiles to produce additional C-6 derivatives. A preferred nucleophile is a thiol compound, especially a heterocyclic thiol. Modification of an azido group at the 6position is performed, e.g., by reducing the azido group to an amino group, which in turn is functionalized by means of reductive alkylation, nucleophilic substitution, or other amino-group reactions well known to those skilled in the art. These approaches are illustrated in many examples. In a preferred embodiment of the invention, an azido group is partially reduced by reaction with a phosphine compound to produce an iminophosphorane.

The synthesis of the target compound is completed by removing any protecting groups that may be present in the penultimate intermediate using standard techniques that are well known to those skilled in the art. The deprotected final product is then purified, as necessary, using standard techniques such as ion exchange chromatography, reverse phase HPLC, MPLC on reverse phase polystyrene gel and the like, or by recrystallization.

The final product may be characterized structurally by standard techniques such as NMR, IR, MS and UV. For ease of handling, the final product, if not crystalline, may be lyophilized from water to afford an amorphous, easily handled solid.

In general, introduction of the R¹ group is preferably by reductive amination, either directed or to a peptide-protective species. In general, introduction of the R² group is preferably by azide displacement/reduction, amine nucleophilic displacement, and/or acylation.

More specifically, the amine substituents of vancomycin are protected while introducing a functional group such as mesitylenesulfonyl at the C₆ position. The allyloxycarbonyl protective groups are introduced by reaction of a vancomycin hydrochloride in aqueous solution with N-(allyloxycarbonyloxy) succinimide contained in an organic solvent such as acetone. A preferred procedure is to treat the aqueous solution of vancomycin with the organic solvent solution of the succimide.

The protected solid product is reacted with an alkyl halide reactant such as alkyl bromide in the presence of an alkali metal bicarbonate to form an alkyl ester of the carboxyl and protect that position. The resulting alkyl ester is then reacted with a compound which will introduce a functional group such as mesitylenesulfonyl chloride in a solvent such as pyridine so as to introduce the mesitylenesulfonyl moiety at the C₆ hydroxy. This compound is then reacted with an alkali metal halide such as KI to introduce I at the C₆ position. The protective alkyl groups is then removed conventionally such as with palladium compound and a phosphinobutane.

This intermediate can be reacted with allyloxycarbonyl succinimide to protect the secondary nitrogen while leaving the primary nitrogen unprotected. This intermediate can be reacted with an aldehyde such as benzyloxyaldehyde to introduce a benzyloxy benzyl group at the vancosamine nitrogen. Similarly, other aldehydes can be reacted with the same or similar intermediates to form other derivatives such as R₁CHO aldehydes.

After the vancosamine is suitably substituted, the intermediate is reacted with an alkali azide to form an azide which is then reacted with a phosphine for conversion to the amine. Obviously substituted amines such as the substituents shown in this position can be provided using these reactions. After the vancosamine amine polar group is introduced, the protected secondary amine is deprotected to produce the final product.

The compounds of the present invention are valuable antibacterial agents active against various gram-positive and, to a lesser extent, gram-negative bacteria. Accordingly, these compounds find utility in human and veterinary medicine.

Many of compounds of the present invention are biologically active against VRE/MRSA/MRCNS. In vitro antibacterial activity is generally predictive of in vivo activity. It is contemplated that the compounds of the present invention will be administered to a mammal infected with a susceptible bacterial organism.

Using standard susceptibility tests, the compounds of the invention are determined to be active against VRE/MRSA.

The compounds of the invention can be formulated in pharmaceutical compositions by combining the compound with a pharmaceutically acceptable carrier. Examples of such carriers are set forth below.

The compounds may be employed in powder or crystalline form, in liquid solution, or in suspension. They may be administered by a variety of means; those of principal interest include: topically, orally and parenterally by injection (intravenously or intramuscularly).

Compositions for injection, a preferred route of delivery, may be prepared in unit dosage form in ampoules, or in multidose containers. The injectable compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline, or another injectable liquid, e.g., peanut oil for intramuscular injections. Also, various buffering agents, preservatives and the like can be included.

Topical applications may be formulated in carriers such as hydrophobic or hydrophilic base formulations to provide ointments, creams, lotions, in aqueous, oleaginous, or alcoholic liquids to form paints or in dry diluents to form powders.

Oral compositions may take such forms as tablets, capsules, oral suspensions and oral solutions. The oral compositions may utilize carriers such as conventional formulating agents and may include sustained release properties as well as rapid delivery forms.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antibacterial arts. Another factor influencing the precise dosage regimen, apart from the nature of the infection and peculiar identity of the individual being treated, is the molecular weight of the compound.

The compositions for human delivery per unit dosage, whether liquid or solid, may contain from about 0.01% to as high as about 99% of active material, the preferred range being from about 10-60%. The composition will generally contain from about 15 mg to about 2.5 g of the active ingredient; however, in general, it is preferable to employ dosage amounts in the range of from about 250 mg to 1000 mg. In parenteral administration, the unit dosage will typically include the pure compound in sterile water solution or in the form of a soluble powder intended for solution, which can be adjusted to neutral pH and be made isotonic.

The invention described herein also includes a method of treating a bacterial infection in a mammal in need of such treatment comprising administering to said mammal a compound of the invention in an amount effective to treat said infection.

The preferred methods of administration of the antibacterial compounds of the invention include oral and parenteral, e.g., i.v. infusion, i.v. bolus and i.m. injection.

These compounds exhibit desirable levels of antibiotic activity when tested against a panel of bacterial strains, including certain vancomycin-resistant strains, as described in greater detail, below. The compounds of the invention consistently provide an increase in activity to vancomycin and teicoplanin.

In Vitro Assay Utilizing a Panel of Bacterial Strains

Assay Protocol

National Committee for Clinical Laboratory Standards (NCCLS) guidelines [Methods for Dilution Antimicrobial Susceptibility Tests for Bacteria that Grow Aerobically—Fourth Edition; Approved Standard (NCCLS Document M7-A4, January 1997)] are used to establish the assay, although variations are made to optimize the published method according to the specific needs of the present investigation.

The test panel currently includes eight enterococci and 13 staphylococci, which are selected based upon their antibiotic susceptibility profiles. The methicillin-sensitive Staphylococcus aureus (MSSA) strain MB2985 (Smith isolate) is used to assesses the potential for serum protein binding of the compounds. The paired Gram-negative strains ASP #49 (envA−) and ASP #50 (envA+) (M. Salvatore/N. Lee) are included to judge membrane effects. Table 1 presents a detailed description of the strains included in the panel, including some of the sources of the strains of bacteria.

Relevant antibiotic controls include the glycopeptide antibiotic vancomycin and the penem antibiotic Schering 29482, which exhibits reduced activity in the presence of Human Serum Albumin or Fraction V, due to binding of the antibiotic. The glycopeptide antibiotic teicoplanin is included whenever possible.

Recommended Sources of Supplies

Trypticase Soy Broth (TSB) (Source: BBL, Becton Dickinson Microbiolgy Systems, Cockeysville, Md. 21030, U.S. A.)

Horse Serum (HS) (Source: GIBCO BRL Laboratories, Grand Island, N.Y. 14072, U.S. A.)

Mueller Hinton II Broth (MH II) (Source: BBL, Becton Dickinson Microbiology Systems, Cockeysville, Md. 21030, U.S. A.)

Brain Heart Infusion Broth (BHI) and Brain Heart Infusion Agar (BHI Agar) (Source: Difco Laboratories, Detroit, Mich. 48232, U.S. A.)

Human Serum Albumin, Fraction V (HSA) (Source: Calbiochem Corporation, La Jolla, Calif. 92037, U.S. A.)

Overnight Growth Medium

TSB, containing 10% HS* for vancomycin intermediate S. aureus/methicillin-resistant S. aureus (VISA/MRSA) strains, is inoculated from an appropriate source (frozen broth or agar slant) and grown approximately 17 hr at 35° C. with shaking at 220 rpm. Cultures are grown in tubes with a volume of 5 ml for enterococci and 2 ml for all other strains.

* HS is aseptically added to TSB on top of normal volume of medium at time of use: 0.5 mL HS+5 mL TSB fi TSB+10% HS

Culture Dilution

Four milliliter (4 ml) overnight culture is added to 36 ml physiological saline to obtain tenfold dilutions (enterococci). Overnight culture (0.4 ml) is added to 39.6 ml saline to obtain 100-fold dilutions (all strains except enterococci). Diluted cultures are maintained on ice until time of inoculation of test plates.

Plate Medium for Obtaining Titers of Overnight Cultures

The number of CFU/ml is determined on BHI agar plates, although this is not done routinely because titers of overnight cultures are relatively constant. The test media include:

BHI for enterococci and VISA/MRSA;

MH II (i.e., cation-adjusted Mueller-Hinton broth) for MRS, MSS and E. coli.;

MHII+HSA for MSSA is prepared as follows:

a. 2× MH 11+86 mg/ml HSA

b. Dissolve 4.3 g HSA in 50 ml autoclaved 2× MH II.

c. pH to 7.0 by adding 2M MOPS, sodium.

d. Filter sterilize using 0.22 μm Coming cellulose acetate filter, used because of reported low protein binding.

e. 1× MH II+43 mg/ml HSA

f. Dilute the 2× medium twofold and filter as above.

Preparation of Test Plates

Using a Denley liquid handling system (or similar automatic device), 100 μl× medium is added to each well in columns 2-12 of a 96-well microtiter dish. Using a multichannel pipettor, 100 μl 2× medium is added to each well in column 1. Plates may be filled on the day prior to assay, wrapped in plastic bags and refrigerated.

Preparation of Antibiotics

Vancomycin, Schering 29482, and teicoplanin are prepared on a weight per volume basis using 10 mM 3-(N-morpholino)propane-sulfonic acid (MOPS) buffer pH 7. Test compounds are received in solution in appropriate solvent (typically as 1 mg/ml in DMSO) or are dissolved in appropriate solvent prior to further dilution in 10 mM MOPS buffer, pH7. Consistent with NCCLS guidelines, antibiotics are handled aseptically but are not otherwise sterilized.

Assay

100 μl appropriately diluted antibiotic solution is added to the first well of the designated row of the 96-well microtiter dish and serially diluted by twofold across the row using the Denley liquid handling system. With the aid of a Dynatech NEC₂₀₀₀ inoculator, each well of the microtiter dish is inoculated with 1.5 μl diluted overnight culture, yielding approximately 1-5×106 CFU/ml for enterococci and approximately 3-7×105 CFU/ml for all other strains. Dishes are placed in stacks of no more than five, wrapped in plastic bags and incubated at 35° C.

Indication of result(s) type (%INH, IC50, Zone size, etc.)

MIC=minimum inhibitory concentration

Interpretation of the Results on the Basis of Activity

Presence or absence of growth is scored at 18-20 hours for strain MB2985 and for E. coli., at 22-24 hr for all other strains. MIC is defined as the lowest concentration of antibiotic that allows no visible growth following incubation. The compounds of the present invention display adequate improvement over the activity exhibited by the control compounds.

In Vivo Mice Studies Protocol for the Methicillin-sensitive Staphylococcus aureus Septicemia

Selected compounds of the invention are tested in an in vivo mouse model. Single dose subcutaneous antibiotic protection from septicemic infections is measured as described by Gill, C. J., J. J. Jackson, L. Gerckens, B. Pelak, R. Thompson, J. Sundelof, H. Kropp and H. Rosen. Antimicrob. Agents Chemother. 42:1996-2001 (1998). Survival is monitored for seven days. ED₅₀'s and LD₅₀'s are determined by the method of Knudsen and Curtis. J. Am. Stat. Assoc. 42:282-296 (1947). Septicemia is induced in 20 gram ICR (derived from CD-1) female mice by intraperitoneal infection with Staphylococcus aureus strain MB2985. Infection is given i.p. in Brain Heart Broth (BHB) at an infectious inoculum of 1.8×10⁷ cfu/mouse. Drug is administered subcutaneously immediately after the infection is initiated.

The MIC is determined by microdilution in Mueller-Hinton broth (MHB) according to the National Committee for Clinical Laboratory Standards guidelines after incubation for 24 hours. Enterococci are tested in cation-supplemented Mueller-Hinton broth at 1.4×10⁵ cfu/ml. MIC is defined as the lowest concentration of antibiotic, which inhibits visible growth.

TABLE 1
Table 3, Bacterial Strains for Assays

| | | Phenotype^{b} | | | | |
|---|---|---|---|---|---|---|
| Strain | | Van^{c} | Gent | Amp | Ipm | Source |
| Enterococci | | | | | | |
| E. faecium | RLA1 | S | S | R | R | Dr. B. Murray, Houston, TX |
| E. faecium | CL 4931 (VanA) | R | R | R | R | NY Hospital, NYC |
| E. faecium | CL 5053 (VanA) | R | R | R | R | Bellevue Hospital, NYC |
| E. faecium | CL 5242 (VanA) | R | R | R | R | Dr. Sahm, Wash. Univ. School of Med. |
| E. faecalis | MB2864 | S | S | S | S | Merck Clinical Culture Collection |
| E. faecalis | CL 4877 (VanB) | R | R | S | S/I | Univ. of Maryland Hospital, Baltimore, MD |
| E. faecalis | CL 5244 (VanB) | R | S | S | S | Merck Clinical Culture Collection |
| E. gallinarum | CL 4886 (VanC) | I | R | S | S | Dr. Shlaes, Cleveland VA Hospital |
| Staphylococci | | | | | | |
| MSSA | MB2985^{a} | S | S | S | S | Mouse pathogen (Smith strain) |
| MRSA | CL 3033 | S | | R | R | VA Hospital, East Orange, NJ |
| MRSA | COL | S | S | R | R | Dr. A. Tomasz, Rockefeller Univ., NYC |
| MRSA | MH 76 | | | | | |
| VISA/MRSA | CL 5705 | S | R | R | R | Mu 3; Dr. K. Hiramatsu, Juntendo Univ., Japan |
| VISA/MRSA | CL 5706 | I | R | R | R | Mu 50; Dr. K. Hiramatsu, Juntendo Univ., Japan |
| MRCNS (S. epi.) | CL 3069 | S | | | R | Univ. of Texas |
| MRCNS (S. hom.) | CL 227 | S | | | R | Temple Univ. Hosp., Philadelphia, PA |
| MRCNS (S. haem.) | CL 171 | S | | | R | Temple Univ. Hosp., Philadelphia, PA |
| MRCNS (S. haem.) | CL 202 | S | | | R | Temple Univ. Hosp., Philadelphia, PA |
| MRCNS (S. hom.) | CL 546 | S | | | R | Wilmington Med. Center, Delaware |

| Gram-negative strains | | | |
|---|---|---|---|
| E. coli (envA⁻) | ASP #49 | R | Tet^{R} envA⁻MB2884 (M. Salvatore/N. Lee) |
| E. coli (envA⁺) | ASP #50 | R | Tet^{R} envA⁺MB2884 (M. Salvatore/N. Lee) |

^{a}Selected for testing in presence of Human Serum Albumin, Frac. V.
^{b}Minimum Inhibitory Concentration (MIC) Interpretive Standards (μg/ml) (Excerpted from NCCLS. Jan. 1998. Vol. 18 No. 1. Document M7-MIC, Tables 2A, 2C and 2D.)

| Antibiotic | Susceptible | Intermediate | Resistant |
|---|---|---|---|
| Vancomycin (all but | ≦4 | 8-16 | ≧32 |
| Enterobacteriacae) | | | |
| Gentamicin (enterococci) | ≦500 | — | >500 |
| Gentamicin (all but enterococci) | ≦4 | 8 | ≧16 |
| Ampicillin (enterococci) | ≦8 | — | ≧16 |
| Ampicillin (staphylococci) | ≦0.25 | — | ≧0.5 |
| Ampicillin (Enterobacteriaceae) | ≦8 | 16 | ≧32 |
| Imipenem (all but enterococci) | ≦4 | 8 | ≧16 |

Abbreviations: Van, vancomycin; Gent, gentamicin; Amp, ampicillin; Ipm, imipenem

The following examples are presented to illustrate the invention but it is not considered to be limited thereto. Parts are by weight unless otherwise indicated.

HPLC Conditions for Examples 1-19:

Analytical HPLC:

Instrument: Hewlett Packard 1090 Liquid Chromatograph

Column: YMC Combiscreen ODS-A, 50×4.6 mm

Conditions: 10% acetonitrile+0.1% (v/v) TFA: 90% H₂O+0.1% (v/v) TFA to 100% acetonitrile+0.1% TFA over 4 minutes (linear gradient)

Flow rate=4 mL/min

Preparative HPLC:

Instrument: Rainin Dynamax Liquid Chromatograph

Column: YMC Combiprep ODS-A, 20×100 mm

Conditions: 10% acetonitrile+0.1% (v/v) TFA: 90% H₂0+0.1% (v/v) TFA to 100% acetonitrile+0.1% TFA over 15 minutes (linear gradient)

Flow rate=20 mL/min

### EXAMPLE 1

Morpholinylamido-N-4-(3-4-dichlorobenzyloxy)benzyl-glucose-6-deoxy-6-aminovancomycin

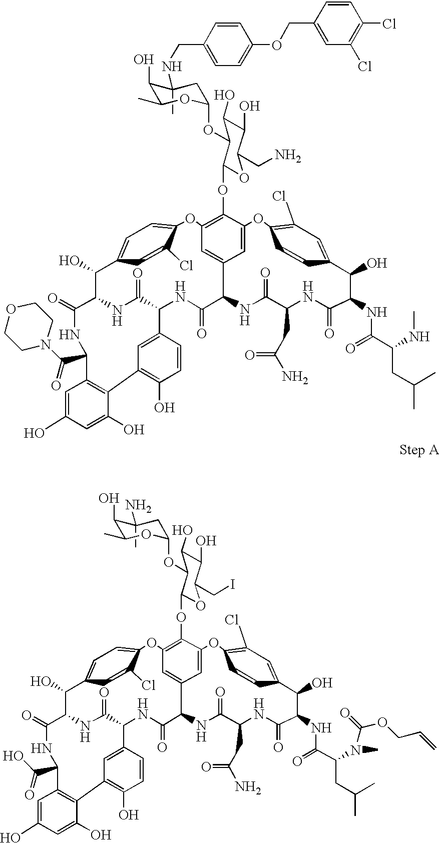

N′-allyloxycarbonyl-glucose-6-deoxy-6-iodovancomycin

To a stirring solution of glucose-6-deoxy-6-iodovancomycin (1.0 mmol, 1.56 g) and NaHCO₃ (4 mmol, 336 mg) in 40 mL of 1:1 dioxane/H₂O cooled in an ice bath was added dropwise a solution of N-(allyloxycarbonyloxy)succinimide (1.0 inmol, 199 mg) in 4 mL of dioxane. The reaction was stirred at 4° for 65 h. 20 mL of isopropanol was added to the reaction mixture, and the solvent removed by rotary evaporation. The solid residue was triturated in CH₂Cl₂, filtered and dried under high vacuum affording 1.85 g of crude solid. HPLC retention time=1.24 min.

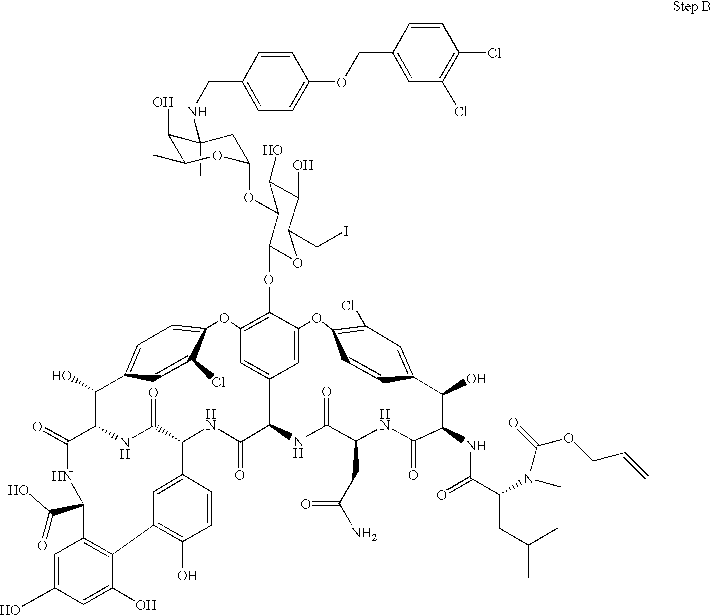

N-4-(3-4-dichlorobenzyloxy)benzyl-N′-allyloxycarbonyl-glucose-6-deoxy-6-iodovancomycin

A solution of the product from Step A (0.5 mmol, 767 mg) and 4-(3-4-dichlorobenzyloxy)benzaldehyde (1.5 mmol, 422 mg) was heated at 50 in 20 mL of DMF containing 2% (v/v) of HOAc for 2 h. Solid NaBH₃CN (3.5 mmol, 220 mg) was added to the solution, which was heated at 50° for 18 h. The product was precipitated by addition of 150 mL of Et₂O. The solid was filtered, washed with Et₂O and CH₂Cl₂ and dried in vacuo, affording 773 mg of solid. HPLC retention time=1.98 min.

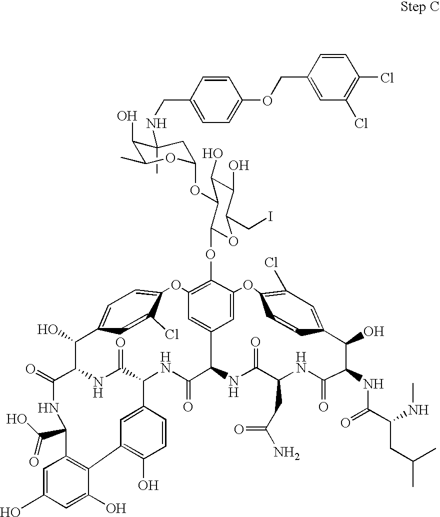

N-4-(3-4-dichlorobenzyloxy)benzyl-glucose-6-deoxy-6-iodo-vancomycin

To the product from Step B (0.42 mmol, 760 mg) in 11 mL of 10:1 DMF/piperidine was added a 3 mL solution of Pd₂dba₃ (0.01 mmol, 10 mg) and 1,4-bis-(diphenylphosphino)butane (0.025 mmol, 11 mg) in THF under nitrogen atmosphere. After 30 min. the product was precipitated by addition of 30 mL of Et₂O. The solid was filtered, washed with Et₂O and CH₂Cl₂ and dried in vacuo, affording 741 mg of slightly greenish solid. HPLC retention time=1.46 min.

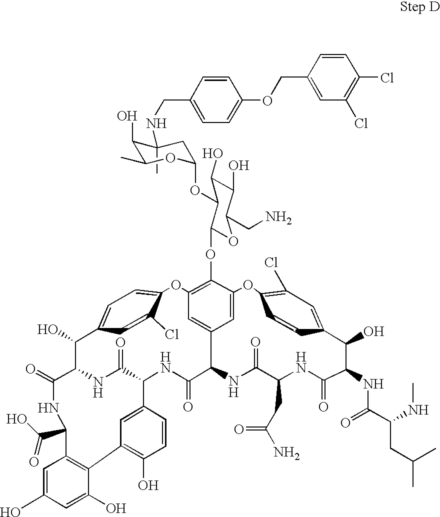

N-4-(3-4-dichlorobenzyloxy)benzyl-N′-allyloxycarbonyl-glucose-6-deoxy-6-amino-vancomycin

A slurry of the product from Step C (0.27 mmol, 500 mg) and NaN₃ (3 mmol, 200 mg) were stirred in 7 mL of DMF at 60° for 18 h. The reaction was allowed to reach ambient temperature, and the solution was filtered to remove solid NaN₃. The product was precipitated by addition of 50 mL of Et₂O to the filtrate. The solid was filtered, washed with Et₂O and CH₂Cl₂and dried in vacuo. The solid was then triturated in 20 mL of H₂O , filtered and dried in vacuo. The azide (HPLC retention time=1.43 min.) was treated with Ph₃P (0.76 mmol, 200 mg)in 10 mL of 4:1 THF/H₂O for 20 h. at 60°. Solvent was removed by rotary evaporation, and the solid residue was triturated in CH₂Cl₂and filtered. The filter cake was washed with CH₂Cl₂ and dried in vacuo affording 352 mg. of crude product. Reverse-phase HPLC purification of 180 mg of the crude product afforded 54 mg of the desired product. HPLC retention time=1.28 min. LC-MS: [M+H]⁺=1711.

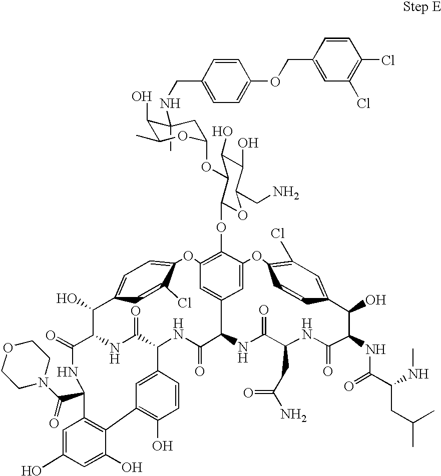

Morpholinylamido-N-4-(3-4-diehlorobenzyloxy)benzyl-glucose-6-deoxy-6-amino-vancomycin

To a stirring solution of crude product from Step D (0.1 mmol, 171 mg), HOBt (0.5 mmol, 77 mg) and morpholine (1.0 mmol, 87 mg) in 4 mL of DMF cooled in an ice bath was added dropwise a 1 mL solution of PyBOP (0.14 mmol, 73 mg) in DMF. After 1 h. the product was precipitated by addition of 10 mL of Et₂O. The solid was washed with Et₂O and CH₂C₂ and dried. The product was purified by reverse phase HPLC affording 40 mg of the titled compound. HPLC retention time=1.31 min; Mass Spec. [M+H]⁺1781.

### EXAMPLE 2

Hydroxyethoxyethylamido-N-4-(3-4-dichlorobenzyloxy)benzyl-glucose-6-deoxy-6-amino-vancomycin

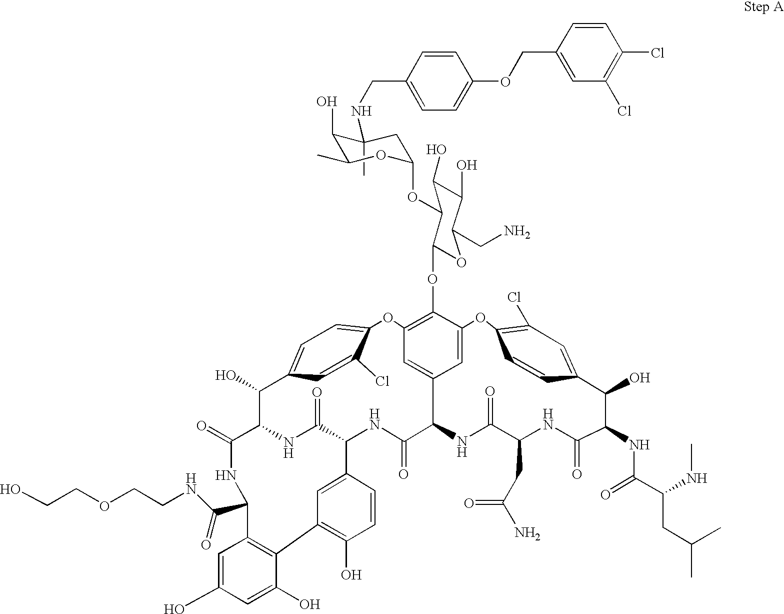

Hydroxyethoxyethylamido-N-4-(3-4-dichlorobenzyloxy)benzyl-glucose-6-deoxy-6-amino -vancomycin

To a solution of HPLC-purified the product from Example 1 Step D (0.01 mmol, 20 mg), HOBt (0.05 mmol, 8 mg) and hydroxyethoxyethylamine (0.1 mmol, 10 mg) in 1 mL of DMF cooled in an ice bath was added a 0.2 mL solution of PyBOP (0.0 15 mmol, 8 mg) in DMF. After 30 min. the product was precipitated by addition of 5 mL of Et₂O. The solid was filtered and washed with Et₂O and CH₂Cl₂ and dried. The product was purified by reverse-phase HPLC. HPLC retention time=1.24 min; Mass Spec. [M+H]⁺1799.

### EXAMPLES 3, 4, 5, AND 6

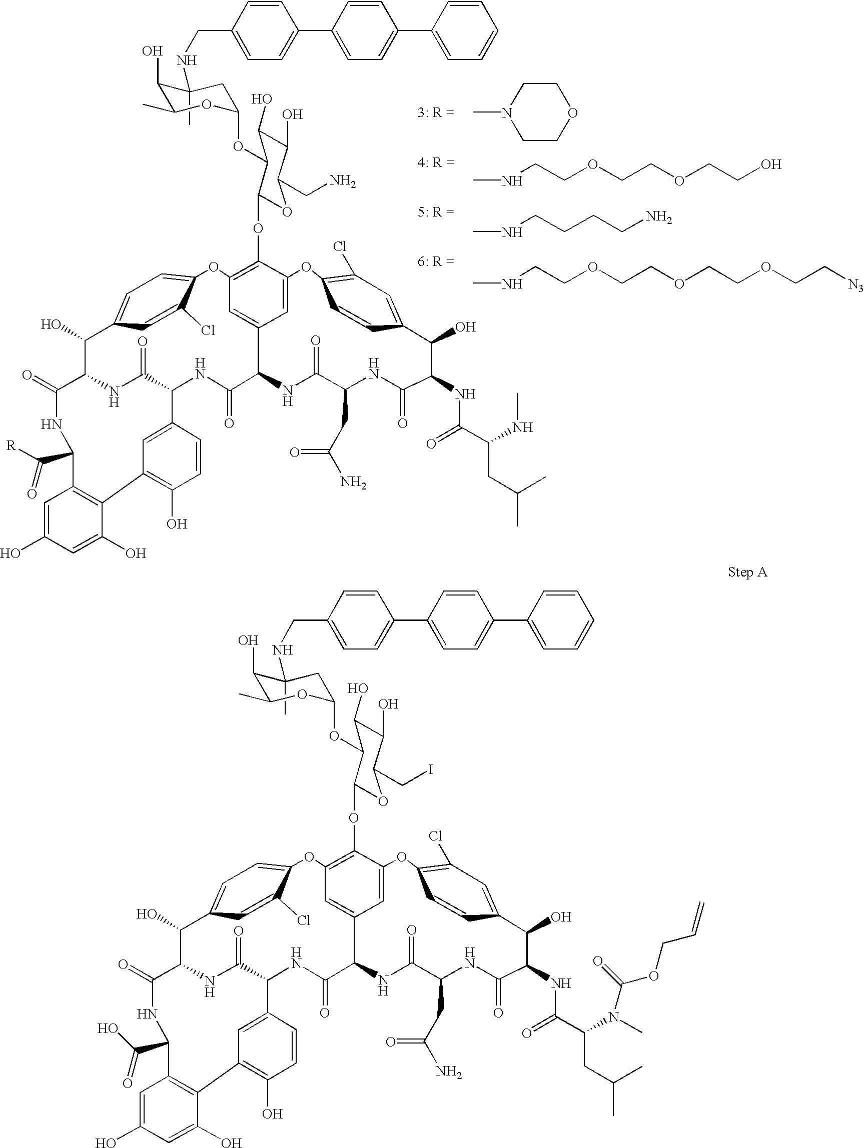

N-4-p-biphenylbenzyl-N′allyloxycarbonyl-glucose-6-deoxy-6-iodovancomycin

A solution of the product from Example 1 Step A (0.1 mmol, 153 mg) and 4-p-biphenylbenzaldehyde (1.5 mmol, 422 mg) was heated at 50° in 4 mL of DMF containing 2% (v/v) of HOAc for 2 h. Solid NaBH₃CN (0.7 mmol, 44 mg) was added, and the solution was heated at 50° for 18 h. The product was precipitated by addition of 40 mL of Et₂O. The solid was filtered, washed with Et₂O and CH₂Cl₂and dried in vacuo, affording 156 mg of solid. HPLC retention time=1.92 min.)

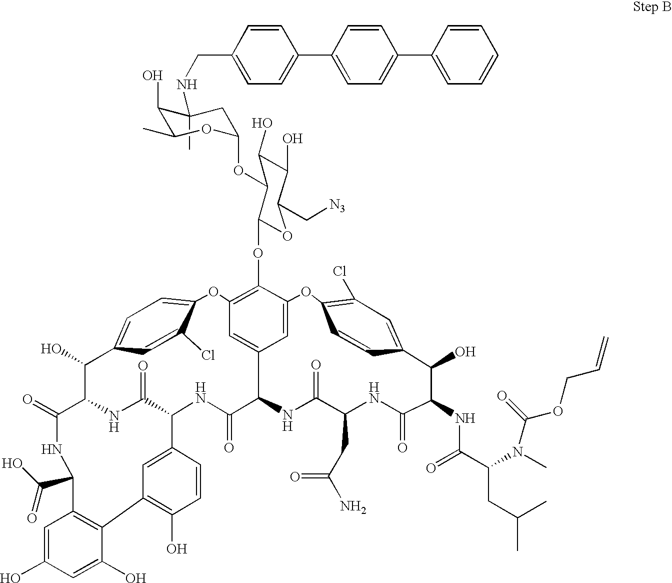

N-4-(3-4-dichlorobenzyloxy)benzyl-N′-allyloxycarbonyl-glucose-6-deoxy-6azido-vancomycin

A slurry of the product from Step A (0.1 mmol, 156 mg) and NaN₃ (1.5 mmol, 100 mg) was stirred in 3 mL of DMF at 80° for 5 h. The reaction was allowed to cool to ambient temperature, and the solution was filtered to remove solid NaN₃. The product was precipitated from the filtrate by addition of 30 mL of Et₂O. The solid was filtered, washed with Et₂O and CH₂Cl₂and dried in vacuo, affording 155 mg of solid.

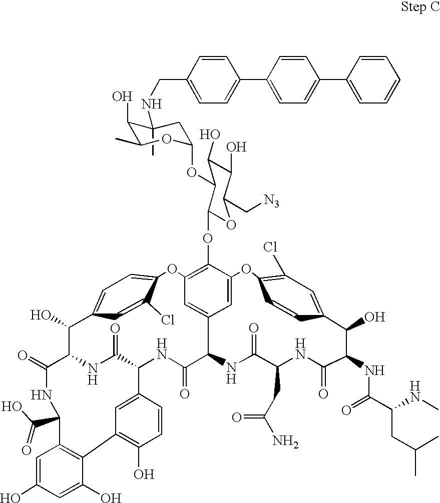

N-4-(3-4-dichlorobenzyloxy)benzyl-glucose-6-deoxy-6-azido-vancomycin

To a solution of the product from Step B (0.04 mmol, 80 mg) dissolved in 2 mL of DMF was added a solution of Pd2dba3 (0.003 mmol, 3 mg) and 1,4-bis (diphenylphosphino)butane (0.006 mmol, 3 mg) in THF under nitrogen atmosphere. After 30 min. the product was precipitated by addition of 30 mL of Et₂O. The solid was filtered, washed with Et₂O and CH₂Cl₂ and dried in vacuo affording 77 mg of slightly greenish solid. HPLC retention time=1.48 min.

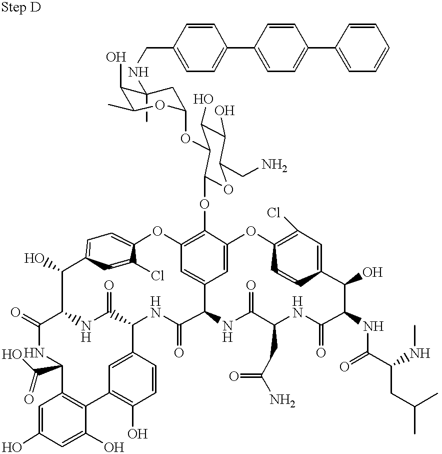

N-4-(3-4-dichlorobenzyloxy)benzyl-glucose-6-deoxy-6-amino-vancomycin

A solution of the product from Step C (0.04 mmol, 77 mg) and Ph₃P (0.19 mmol, 50 mg) in 5 mL of 4:1 THF/H₂O was heated for 16 h. at 60. Solvent was removed by rotary evaporation, and the solid residue was triturated in CH₂Cl₂and filtered. The filter cake was washed with CH₂Cl₂ and dried in vacuo affording 69 mg. of crude product. Reverse-phase HPLC purification of 9 mg of the crude product afforded 1.5 mg of pure product. HPLC retention time=1.32 min. LC-MS: [M+H]⁺=1689.

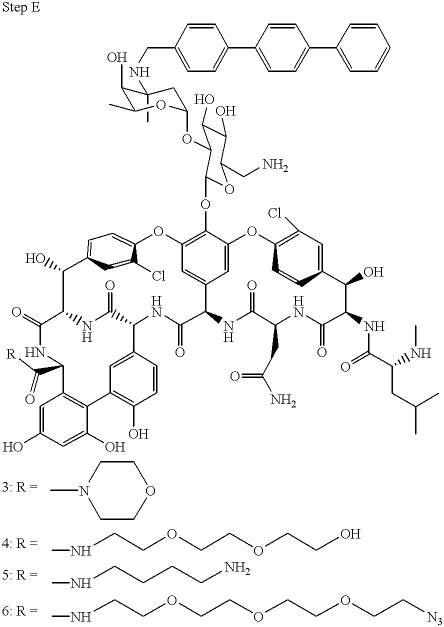

Amide species 3,4,5, and 6 were synthesized using an analogous procedure as follows: to a solution of the product from Step D (0.006 mmol, 10 mg), HOBt (0.03 mmol, 5 mg) and amine (0.1 mmol) in 0.4 mL of DMF cooled in an ice bath was added a solution of PyBOP (0.01 mmol, 5 mg) in 0.2 mL of DMF. After 30 min. the product was precipitated with 5 mL of Et₂O. The solid was filtered, washed with Et₂O and dried. The products were purified by reverse-phase HPLC.

3: HPLC retention time 1.35 min; LC-MS [M+H]⁺ 1759

4: HPLC retention time=1.32 min; LC-MS [M+H]⁺ 1821

5: HPLC retention time=1.20 min; LC-MS [M+H]⁺ 1760

6: HPLC retention time=1.32 min; LC-MS [M+H]⁺ 1890

### EXAMPLES 7,8,9, AND10

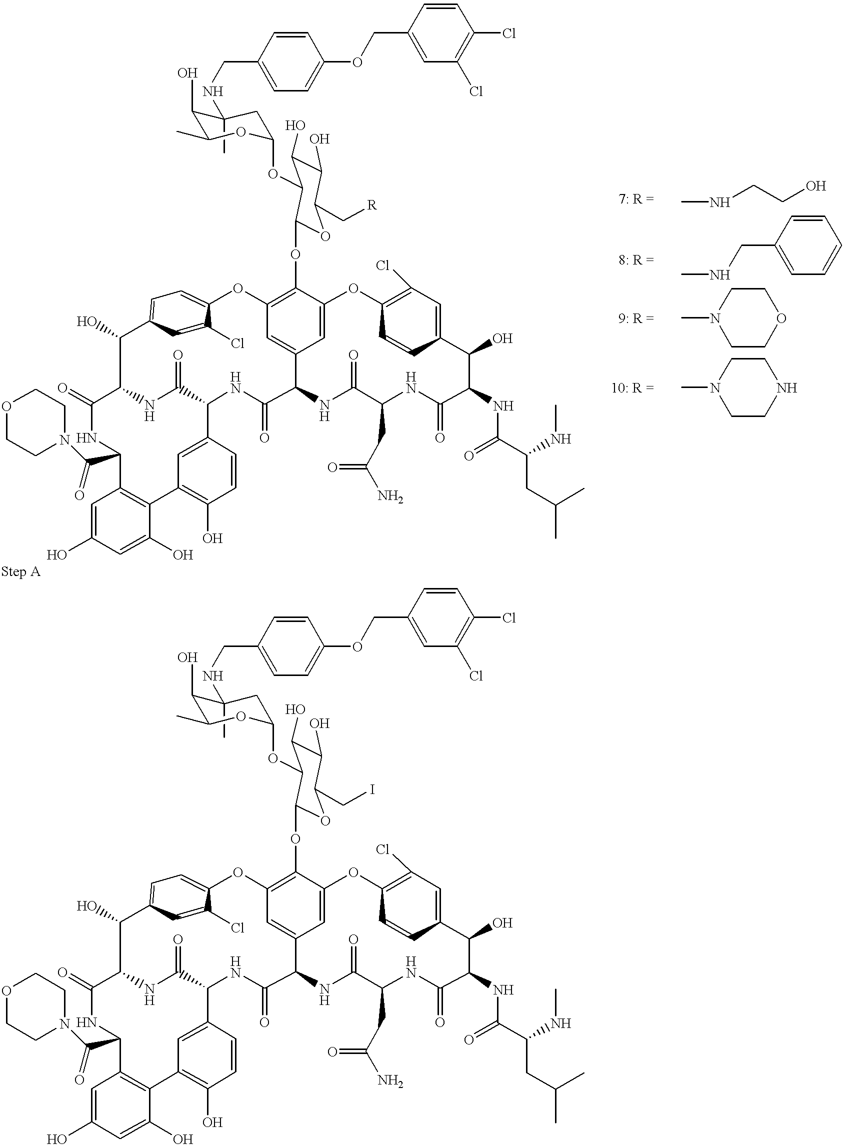

Morpholinylamido-N-4-(3-4-dichlorobenzyloxy)benzyl-glucose-6-deoxy-6-iodo-vancomycin

To a solution of the product from Example 1 Step C (0.1 mmol, 193 mg), HOBt (0.5 mmol, 77 mg) and morpholine (1.0 mmol, 87 mg) in 5 mL of DMF was added solid PyBOP (0.13 mmol, 68 mg). After 40 min. the product was precipitated by addition of 10 mL of Et₂O. The solid was filtered, washed with Et₂O and CH₂Cl₂and dried. The product was purified by reverse phase HPLC affording 37 mg of product. HPLC retention time=1.48 min.

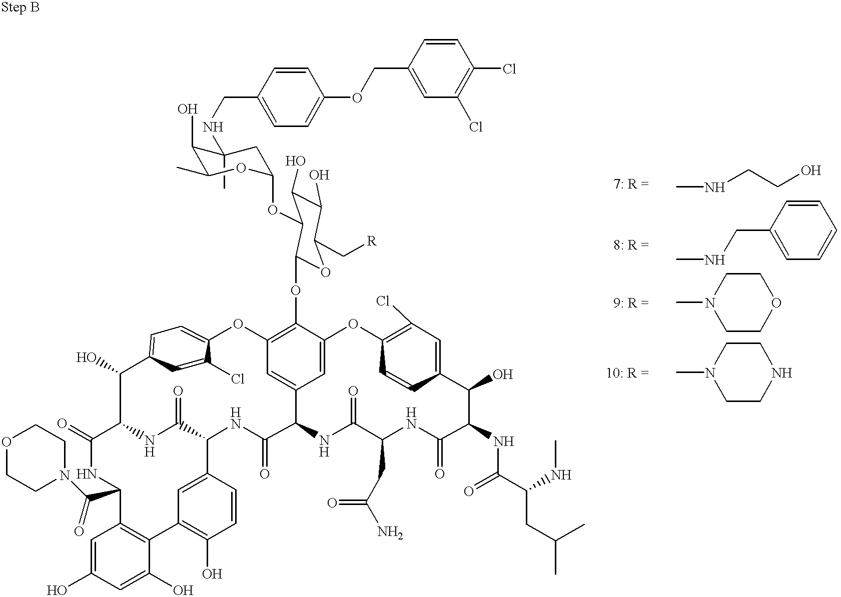

Amine species 7,8,9 and 10 were synthesized using an analogous procedure as follows: a solution of the intermediate from Step A (0.003 mmol, 6 mg) and corresponding amine (100 mg) was heated in 0.5 mL of DMF at 60° for 20 h. The product was precipitated with 7 mL of Et₂O. The solid was centrifuged and the solvent decanted. Products were purified by reverse-phase HPLC.

7: HPLC retention time=1.32 min; LC-MS [M+1H]⁺ 1759

8: HPLC retention time=1.34 min; LC-MS [M+1H]⁺ 1821

9: HPLC retention time=1.32 min; LC-MS [M+1H]⁺ 1760

10: HPLC retention time=1.28 min; LC-MS [M+1H]⁺ 1890

### EXAMPLE 11

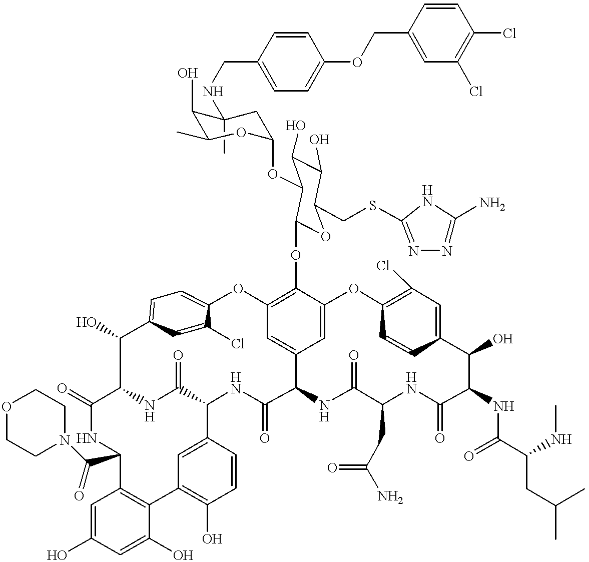

Step A

Morpholinylamido-N-4-(3-4-dichlorobenzyloxy)benzyl-glucose-6-deoxy-6-(3-5amino-1,2,4triazolyl)mercapto-vancomycin

A solution of the product from Example 7 Step A (0.003 mmol, 6 mg) and 5-amino-3-mercapto-1,2,4-triazole (0.04 mmol, 5 mg) was heated in 0.5 mL of DMF at 60 ° for 20 h. The product was precipitated with 7 mL of Et₂O. The solid was centrifuged and the solvent was decanted. The solid was purified by reverse-phase HPLC. HPLC retention time=1.37 min; LC-MS [M+2H]²⁺ 941.

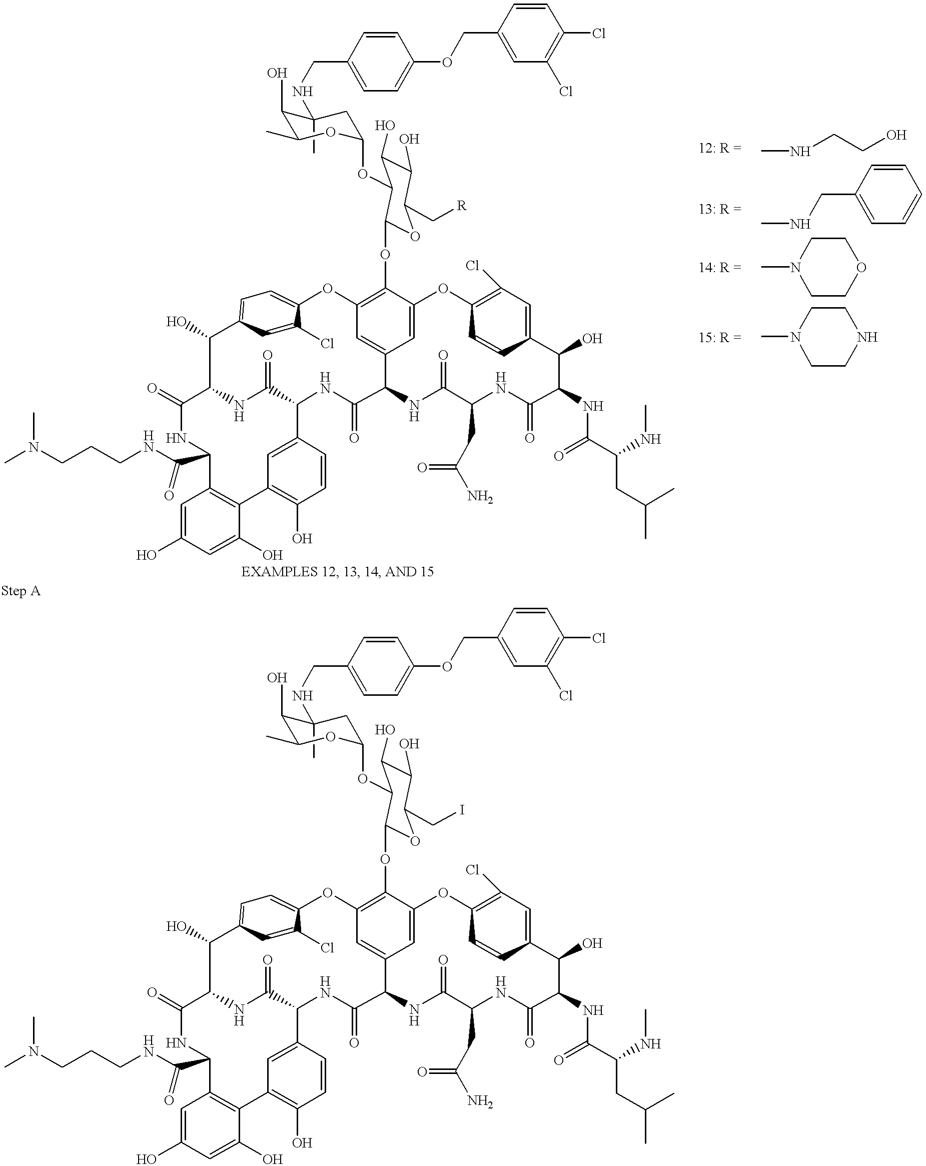

Dimethylaminopropylamido-N-4-(3-4-dichlorobenzyloxy)benzyl-glucose-6-deoxy-6-iodo-vancomycin

To a solution of the product from Example 1 Step C (0.02 mmol, 35 mg), HOBt (0.1 mmol, 15 mg) and 3-dimethylaminopropylamine (0.2 mmol, 20 mg) in 0.7 mL of DMF was added solid PyBOP (0.025 mmol, 13 mg). After 20 min. the product was precipitated by addition of 7 mL of Et₂O. The solid was filtered, washed with Et₂O and dried. affording 36 mg of crude solid. HPLC retention time=1.32 min.

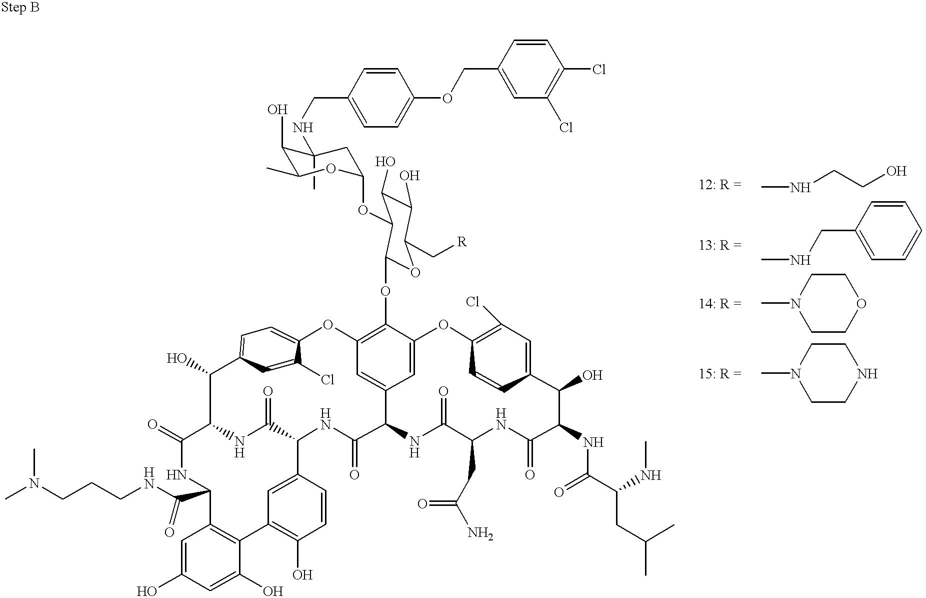

Amine species 12, 13, 14, and 15 were synthesized using an analogous procedure as follows: a solution of the product from Step A (0.003 mmol, 7 mg) and corresponding amine (100 mg) was heated in 0.5 mL of DMF at 60° for 20 h. The product was precipitated with 7 mL of Et₂O. The solid was centrifuged and the solvent decanted. The products were purified by reverse-phase HPLC.

12: HPLC retention time=1.18 min; LC-MS [M+2H]²⁺ 920

13: HPLC retention time=1.20 min; LC-MS [M+2H]²⁺ 943

14: HPLC retention time=1.18 min; LC-MS [M+2H]²⁺ 933

15: HPLC retention time=1.15 min; LC-MS [M+2H]²⁺ 933

### EXAMPLE 16

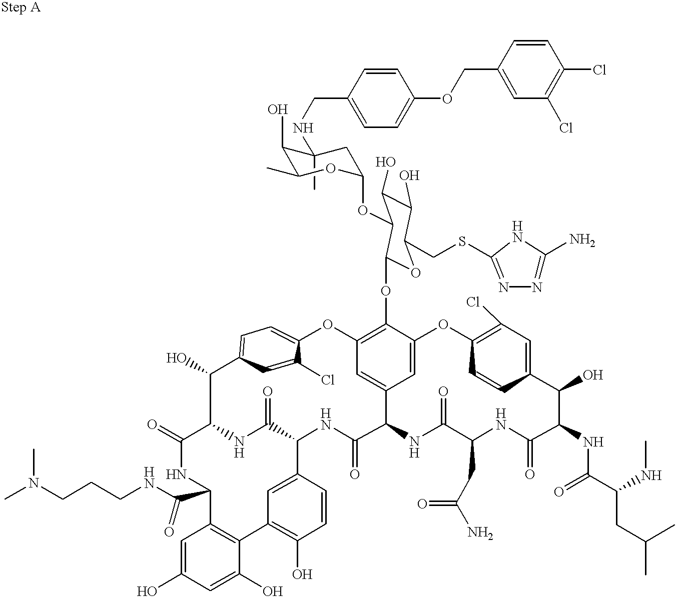

Dimethylaminopropylamido-N-4-(3-4-dichlorobenzyloxy)benzyl-glucose-6-deoxy-6-(3-5amino-1,2,4triazolyl)mercapto-vancomvcin.

A solution of the product from Example 12 Step A (0.003 mmol, 7 mg) and 5-amino-3-mercapto-1,2,4-triazole (0.04 mmol, 5 mg) was heated in 0.5 mL of DMF at 60° for 20 h. The product was precipitated with 7 mL of Et₂O. The solid was centrifuged and the solvent decanted. The solid was purified by reverse-phase HPLC. HPLC retention time=1.23 min; LC-MS [M+2H]²⁺ 948.

### EXAMPLES 17 AND 18

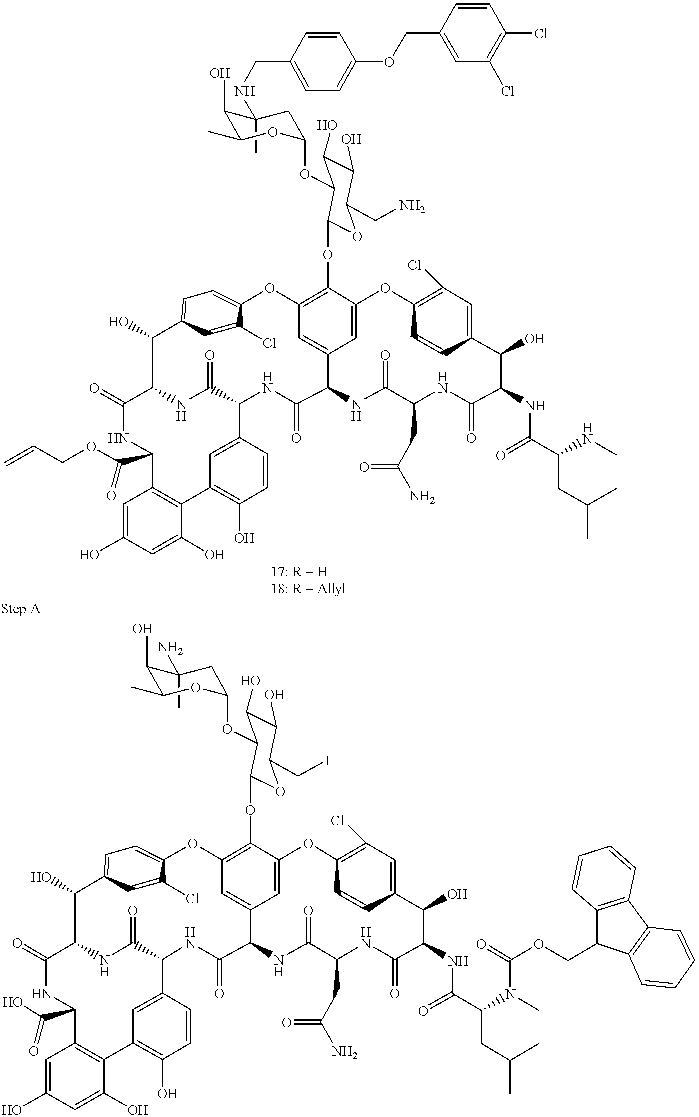

N′-fluorenylmethoxycarbonyl-glucose-6-deoxy-6-iodovancomycin

To a stirring solution of glucose-6-deoxy-6-iodovancomycin (1.0 mmol, 1.56 g) and NaHCO₃ (3 mmol, 252 mg) in 30 mL of 1:1 dioxane/H₂O cooled in an ice bath was added dropwise a solution of of N-(fluorenylmethoxycarbonyloxy)succinimide (1.0 mmol, 337 mg) in 5 mL of dioxane. The reaction was allowed to reach room temperature and stirred for 20 h. 20 mL of isopropanol was added to the reaction mixture, and the solvent was removed by rotary evaporation. The residue was triturated in 100 mL of H₂O . The solid was filtered, washed with 20 mL of H₂O and dried. The product was purified by reverse-phase HPLC, affording 502 mg of product. HPLC retention time 1.24 min.

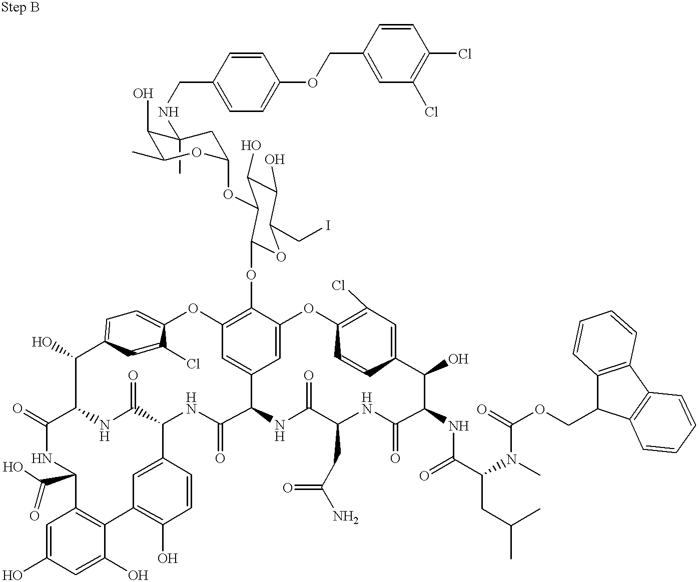

N-4-(3-4-dichlorobenzyloxy)benzyl-N′-fluorenylmethoxycarbonyl-glucose-6-deoxy-6-iodovancomycin

A solution of the product from Step A (0.1 mmol, 170 mg), 4-(3-4-dichlorobenzyloxy)benzaldehyde (0.7 mmol, 196 mg) and NaBH(OAc)₃ (1 mmol, 212 mg) in 3 mL of DMF containing 1.5% HOAc (v/v) was allowed to stand at room temperature. After 16 h, and additional 180 mg of NABH(OAc)₃ was added, and the reaction was allowed to stand 20 h. The product was precipitated by addition of 80 mL of H₂O, filtered, washed with H₂O and dried. The crude product was triturated in CH₂Cl₂, filtered, dried and purified by reverse-phase HPLC, affording 49 mg. of product. HPLC retention time 2.31 min.

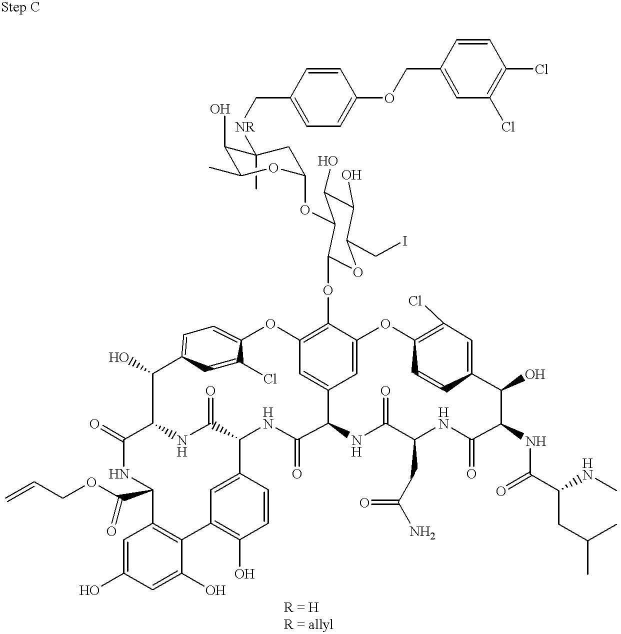

N-4-(3-4-dichlorobenzyloxy)benzyl-glucose-6-deoxy-6-iodo-vancomycin allyl ester and N-allyl-N-4-(3-4-dichlorobenzyloxybenzyl-glucose-6-deoxy-6-iodo-vancomycin allyl ester

To a solution of the product from Step B(0.01 mmol, 21 mg) and allyl bromide (0.05 mmol, 6 mg) in 0.4 niL of DMSO was added NaHCO₃ (0.05 mmol, 4 mg). The reaction mixture was stirred at room temperature for 18 h. The product was precipitated by addition of 7 mL of H₂O . The aqueous phase was decanted and the solid residue was dried. The solid was then taken up in 2 mL of 30% piperidine in DMF (v/v) for 30 min. The deprotected product was precipitated by addition of 10 mL of Et₂O, filtered, washed with Et₂O and dried in vacuo. HPLC analysis indicated the presence of the desired product and the N-allylated sideproduct. HPLC retention times: NH-amine=1.63 min; N allylated=1.74 min.

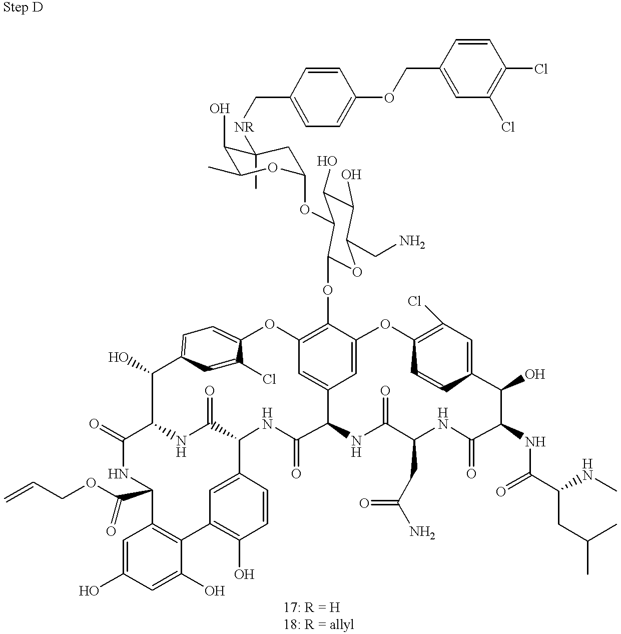

N-4-(3-4-dichlorobenzyloxy)benzyl-glucose-6-deoxy-6-amino-vancomycin allyl ester and N-allyl-N-4-(3-4-dichlorobenzyloxybenzyl-glucose-6-deoxy-6-amino-vancomycin allyl ester

To the products from Step C dissolved in 2 mL of DMF was added NaN₃ (12 mg). The reaction mixture was stirred at 65° for 24 h. The reaction was allowed to reach room temperature and solid NaN₃ was removed by filtration. The filtrate was dried in vacuo and the solid residue was triturated in CH₂Cl₂, filtered and dried. The crude azide and Ph₃P (20 mg) were dissolved in 2 mL of 4:1 THF/H₂O (v/v) and the solution was stirred at 50° for 16 h.

Solvent was removed in vacuo, and the solid residue was triturated in CH₂Cl₂, filtered and dried. Products 17 and 18 were isolated by reverse-phase HPLC. 17HPLC retention time=1.47 min; LC-MS [M+2H]²⁺ 876.18HPLC retention time=1.56 min; LC-MS [M+2H]²⁺ 896.

### EXAMPLE 19

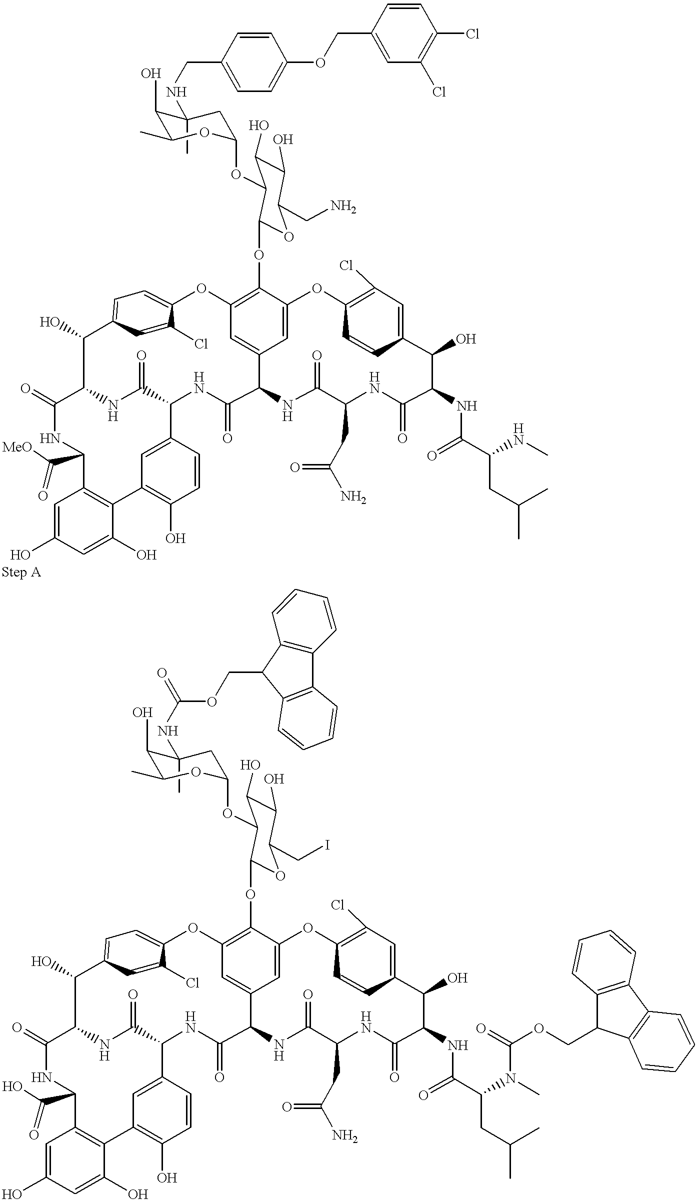

N—N′-bis-fluorenylmethoxycarbonyl-glucose-6-deoxy-6-iodovancomycin

A solution of glucose-6-deoxy-6-iodovancomycin (0.5 mmol, 780 mg), N-(fluorenylmethoxycarbonyloxy)succinimide (1.5 mmol, 506 mg) and NaHCO₃ (2 mmol, 168 mg) in 10 mL of 1:1 dioxane/H₂O was stirred for 16 h. at room temperature. The reaction was stirred for 16 h. 4 mL of isopropanol was added to the reaction, and the solvent was removed by rotary evaporation. The residue was triturated in 20 mL of CH₂Cl₂, filtered and dried. HPLC retention time 2.54 min.

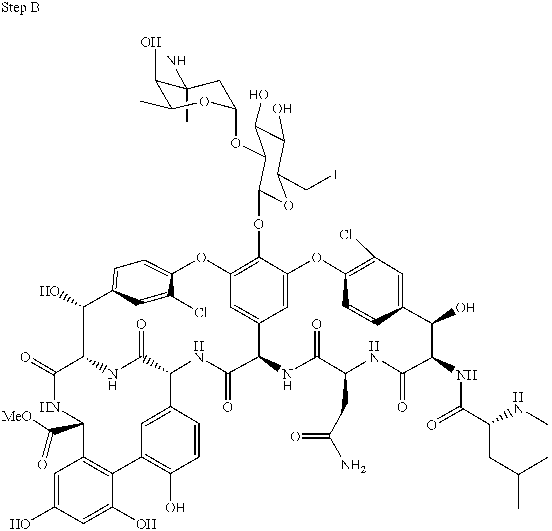

Glucose-6-deoxy-6-iodovancomycin methyl ester

To a solution of the product from Step A (0.5 mmol, 780 mg) and 2,6-lutidine (500 μL) in 10 mL of DMF was added dropwise MeI (500 μL). After 1 h. the reaction mixture was treated with of Et₂O, affording formation of an oily residue. The Et₂O was decanted, and the retentate was treated with 40 mL of H₂O , causing formation of a white solid. The solid was filtered and dried (HPLC retention time of bis-Fmoc species=2.12 min.). The solid was dissolved in 10 mL of 4:1 DMF/piperidine (v/v), and after 15 min. the deprotected product was precipitated by addition of 70 mL of Et₂O. The solid was filtered, washed with Et₂O and CH₂Cl₂ and dried, affording 625 mg. of solid. HPLC retention time=1.61 min.

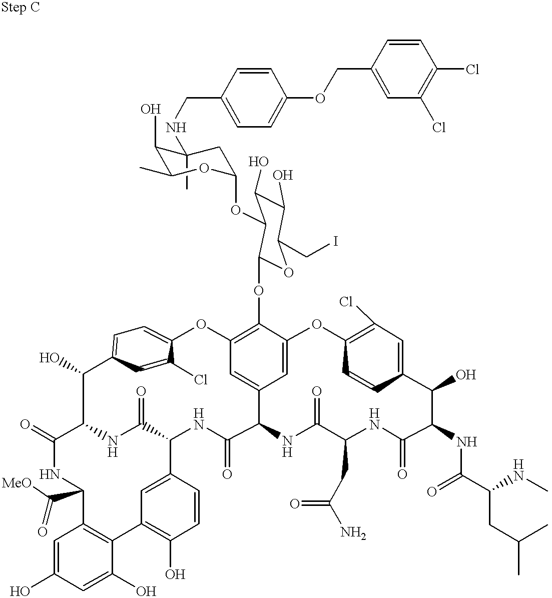

N-4-(3-4-dichlorobenzyloxy)benzyl-glucose-6-deoxy-6-iodovancomycin methyl ester

The product from Step B (0.1 mmol, 157 mg), 4-(3-4-dichlorobenzyloxy)benzaldehyde (0.1 mmol, 28 mg) and Hunig's base (0.5 mmol, 63 mg) were dissolved in 5 mL of dry DMF. The solvent was removed in vacuo. The residue was redissolved in 5 mL of dry DMF and 0.5 mmol of Hunig's base. To the reaction mixture was added a 3 mL solution of NaBH(OAc)₃ (0.9 mmol, 190 mg)in DMF. The reaction was allowed to stand at room temperature for 24 h, after which time the reaction mixture became gelid. The product was precipitated by addition of 50 mL of Et₂O. The solid was filtered, dried and purified by reverse-phase HPLC, affording 17 mg. of 24. HPLC retention time=1.53 min.

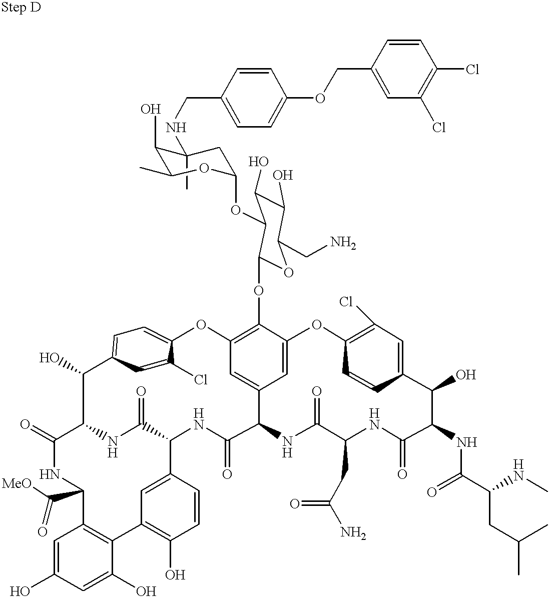

N-4-(3-4-dichlorobenzyloxy)benzyl-glucose-6-deoxy-6-aminovancomycin methyl ester

A solution of the product from Step C (0.01 mmol, 17 mg) and NaN3 (11 mg) were stirred in 1 mL of DMF at 70° for 4 h. The product was precipitated by addition of 20 mL of Et₂O, filtered and dried. The solid was then triturated in 20 mL of H₂O , filtered and dried. The crude azide was heated with Ph₃P (18 mg) in 2 mL of 4:1 THF/H₂O (v/v) at 40° for 20 h. The solvent was removed by rotary evaporation and the solid was triturated in CH₂Cl₂, filtered and dried. Reverse-phase HPLC purification afforded 7 mg of the titled compound. HPLC retention time=1.37 min. LC-MS [M+2H]²⁺ 863.

### EXAMPLE 20

Synthesis of 3″ -N-(4-(3,4-dichlorobenzyloxy)-benzyl)-6′-deoxy-6′-guanidinyl-vancomycin morpholine amide, tris trifluoroacetate salt

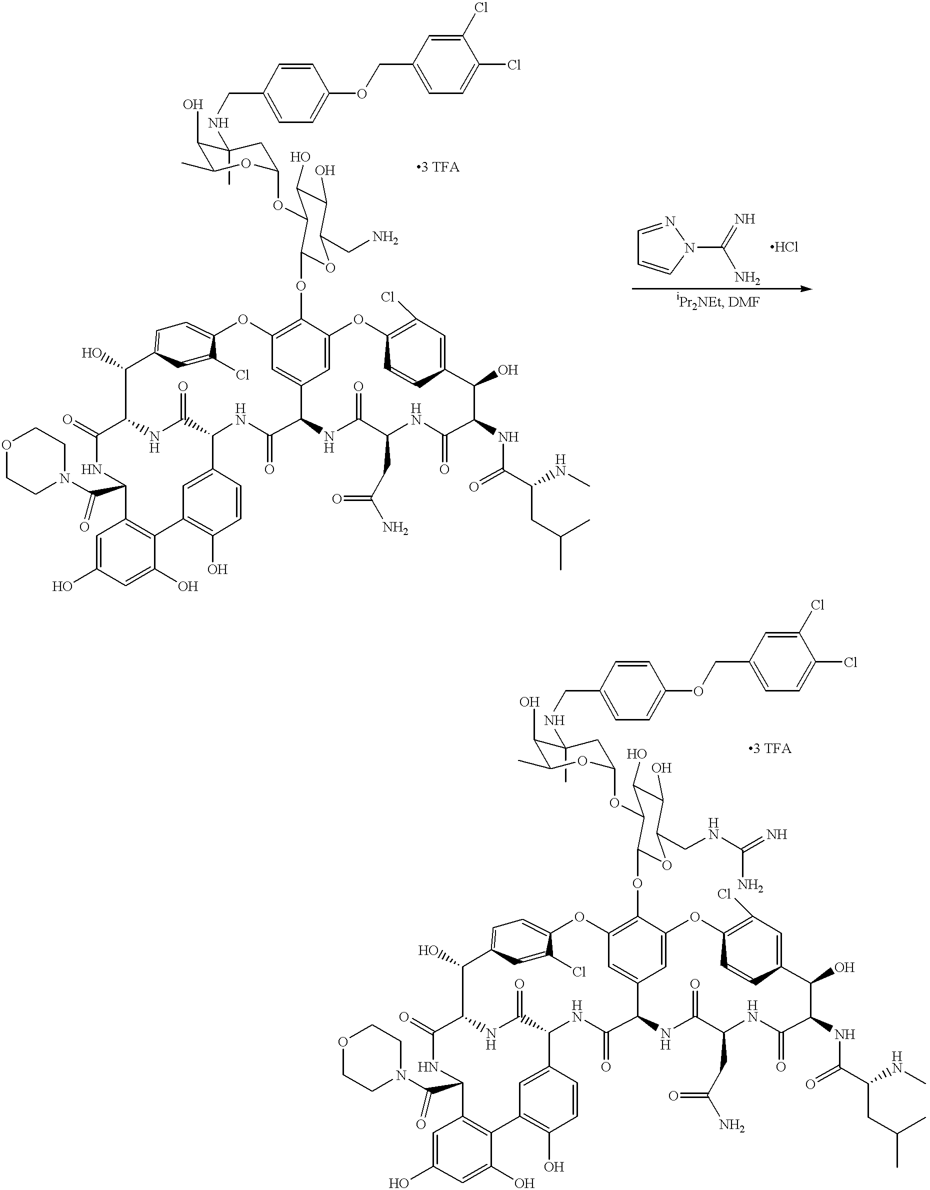

N,N-diisopropylethylamine (0.014 mL, 0.08 mmol) was added to a solution of 3″-N-(4-(3,4-dichlorobenzyloxy)-benzyl)-6′-deoxy-6′-amino-vancomycin morpholine amide, tris trifluoroacetate salt (14 mg, 0.0065 mmol) in anhydrous dimethylformamide (0.50 mL). 1-H-pyrazole-1-carboxamidine hydrochloride (6 mg, 0.04 mmol) was then added and the resulting mixture was stirred at room temperature overnight. After 14 hours, HPLC-MS analysis of the reaction mixture showed a mixture of starting material and product. Additional N,N-diisopropylethylamine (0.014 mL, 0.08 mmol) and 1-H-pyrazole-1-carboxamidine hydrochloride (6 mg, 0.04 mmol) were added and the reaction mixture was again stirred at room temperature overnight. After another 23 hours at room temperature (37 hours total), HPLC-MS analysis of the reaction mixture indicated that most of the starting material was gone. Ether (3.5 mL) was added and the resulting mixture (precipitate formed) was centrifuged. The supernatant was decanted off and discarded. The residual solid was purified by preparative HPLC on a Waters DeltaPak column (C18 100A, 19×300 mm) with gradient elution at 23.7 mL/min from 10:90 acetonitrile:0.1% aqueous triflouroacetic acid to 100% acetonitrile over 15 minutes. The product peak eluted at 8.57 minutes and was collected and evaporated to afford the title compound as a white amorphous solid (12 mg, 84% yield). MS data: m/e 912 ((M+2)/2). Selected ¹H NMR data (500 MHz NMR in pyridine-d5 at 40° C., referenced to internal TMS): δ 6.03 (1H, d, J=4 Hz, H-1″), 5.96 (1H, d, J=8 Hz, H-1″), 5.43 (1H, br q, J=6 Hz, H-5″), 4.90 (2H, s, OCH₂Ar), 4.41 (2H, br s, NCH₂Ar), 4.22 (1H, br s, H-4″), 4.10 & 3.80 (2x 1H, 2 br d, J=13 Hz, H-6′), 3.94 (1H, t, J=9 Hz, H-4′), 3.09 (1H, s, NCH₃), 1.68 (3H, d, J=6 Hz, H-6″).

Analytical HPLC for Examples 21-25 was carried out on a Zorbax SB-C8 (4.6×75 mm, 3.5 um packing) column, with flow of 2 ml/min and the following linear gradient of acetonitrile in water solvent (0.1% TFA): 5% to 60% from 0 to 3 min; 60% to 100% from 3 to 5 min; 100% till 6.5 min; and 100% to 5% from 6.5 to 7 min. Semi-preparative HPLC was done on Zorbax RX-C8 (9.4×250 mm, 5 um packing) column with 7 ml/min flow of acetonitrile/water (0.1% TFA) and appropriate gradient as specified.

### EXAMPLE 21

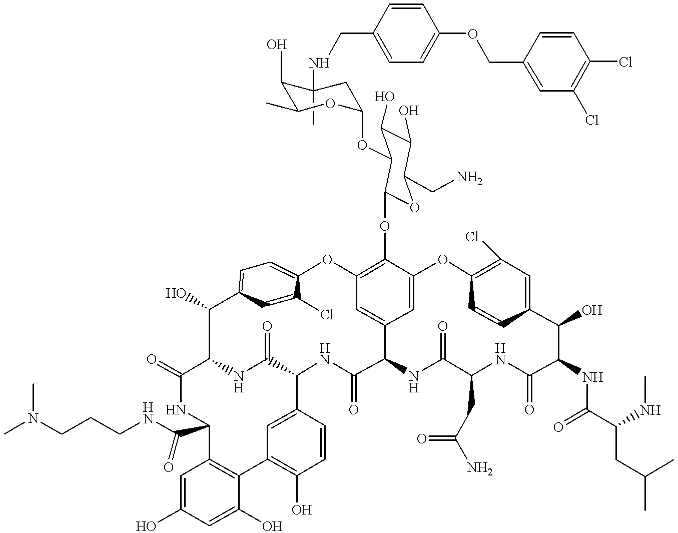

A solution of dimethylaminopropylamine (6 ul, 51 umole) in dried DMF (100 ul) was added to a test tube containing the vancomycin derivative prepared as in Example 1 Step D (7 mg, 3.4 umole), HOBt (2.7 mg, 20 umole) and PyBOP (2.1 mg, 4 umole). The resulting clear solution was stirred for 1 hr at room temperature. The solvent and excess amine were removed by rotary evaporation (water bath kept at 25° C.), and the residue was dissolved in 2 ml of CH3CN:H₂O (1:8, containing ˜1% TFA). Preparative HPLC (acetonitrile in water: 20% to 35% from 0 to 2 min; 35% isocratic till 7 min; 35% to 100% from 7 to 8 min), after lyophilization, afforded 3.3 mg of product, with observed m/e of of 898.4 (doubly charged molecular ion) and analytical HPLC retention time of 3.82 min.

### EXAMPLE 22

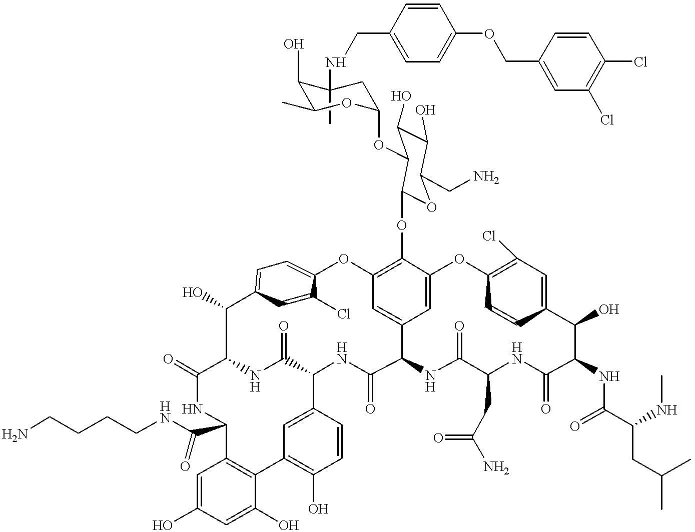

A solution of butanediamine (7 mg, 75 umole) in dried DMF (200 ul) was added to a test tube containing the vancomycin derivative prepared as in Example 1 Step D (15.5 mg, 7.54 umole), HOBt (6 mg, 45 umole) and PyBOP (4.7 mg, 9 umole). A large amount of white solid precipitated out (butanediamine salt). The suspension was stirred for 2 hr at room temperature. The solvent was removed by rotary evaporation (water bath kept at 25° C.), and the residue was dissolved in 2 ml of CH3CN:H₂O (1:4, containing ˜1% TFA). Preparative HPLC (acetonitrile in water: 20% to 32% from 0 to 1 min; 32% isocratic till 7 min; 32% to 100% from 7 to 8 min), after lyophilization, afforded 9.8 mg of product, with observed m/e of 892.5 (doubly charged molecular ion) and analytical HPLC retention time of 3.80 min.

### EXAMPLE 23

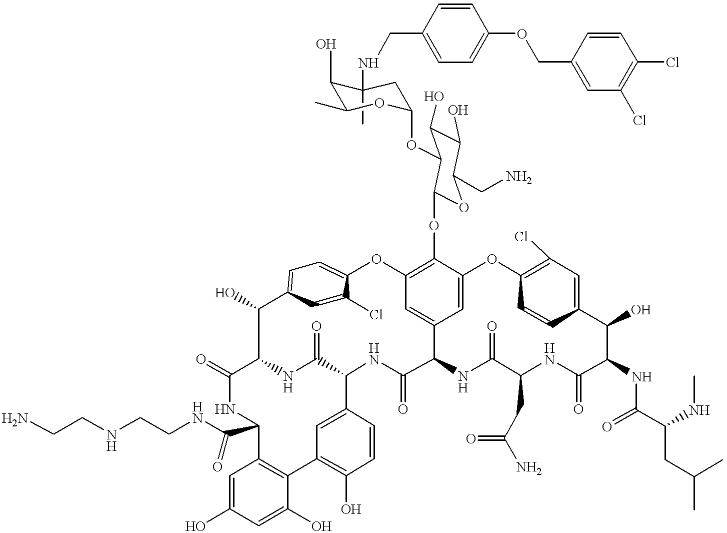

A solution of diethylenetriamine (8 mg, 75 umole) in dried DMF (200 ul) was added to a test tube containing the vancomycin derivative prepared as in Example 1 Step D (15.5 mg, 7.54 umole), HOBt (6 mg, 45 umole) and PyBOP (4.3 mg, 8.3 umole). The resulting clear solution was stirred for 1 hr at room temperature. The solvent was removed by rotary evaporation (water bath kept at 25° C.), and the residue was dissolved in 2 ml of CH3CN:H2O (1:9, containing ˜1% TFA). Preparative HPLC (acetonitrile in water: 20% to 32% from 0 to 1 min; 32% isocratic till 7 min; 32% to 100% from 7 to 8 min), after lyophilization, afforded 2.5 mg of product, with observed m/e of 899.0 (doubly charged molecular ion.)

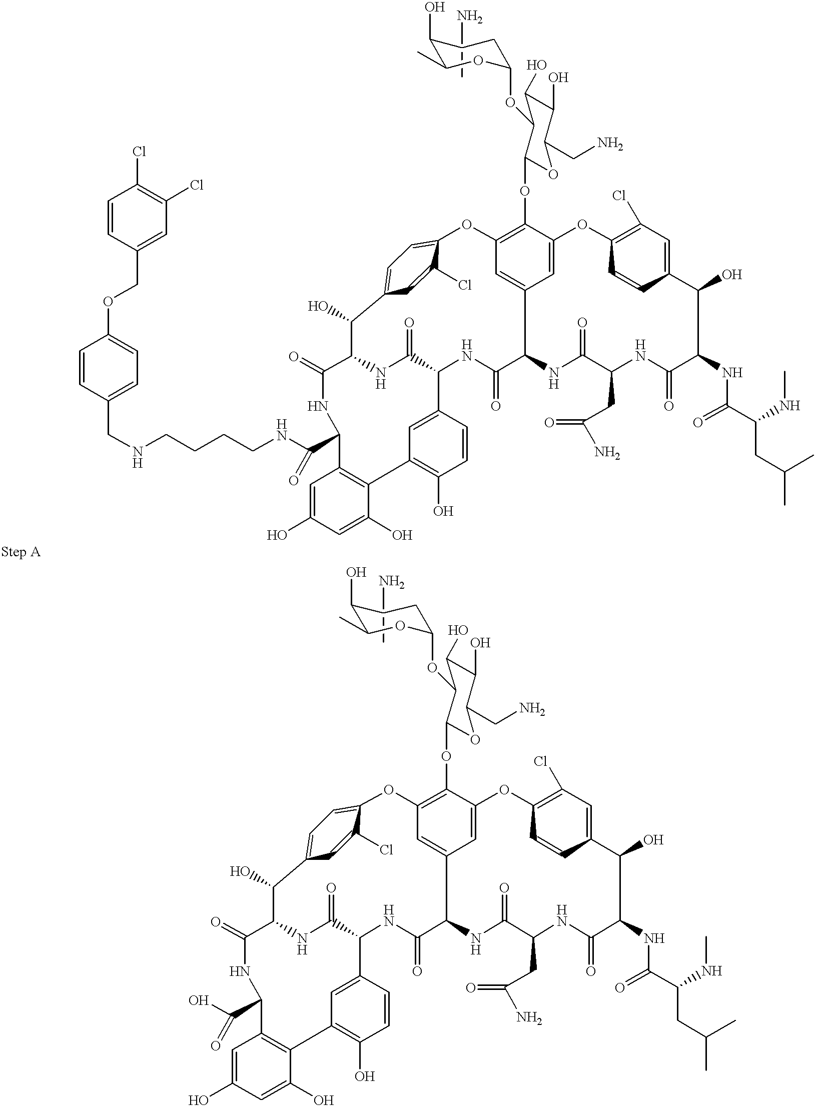

G6-Iodovancosamine (440 mg, 0.25 mmole) was stirred with NaN3 (160 mg, 2.5 mmole) in dried DMF (6 ml, N2 purged before use) at 60° C. for 16 hr. Excess NaN3 was filtered off, and the solution was dropped into ether (200 ml). The solid was collected, washed with more ether, dried in air. This crude product was stirred with PPh3 (320 mg, 1.25 mmole) in 11 ml of THF-H2O (5:1, N2 purged before use) at 60° C. for 20 hr. The solvent was removed on a rotary-evaporator, and the residue dissolved in 7 ml of DMF. This was added to ether (200 ml). The solid was collected, washed with DCM and dried in air. The crude product was dissolved in 13 ml of CH3CN:H2O (1:9, 0.2% TFA) and purified in preparative HPLC (10% to 25% from 0 to 5 min; 25% to 100% from 5 to 5.5 min; 100% from 5.5 min to 7 min; 100% to 10% from 7 to 7.5 min) to give, after lyophiization, 177 mg (40%) of white powder, with observed m/e of 724 (doubly charged molecular ion) and HPLC retention time of 2.80 min.

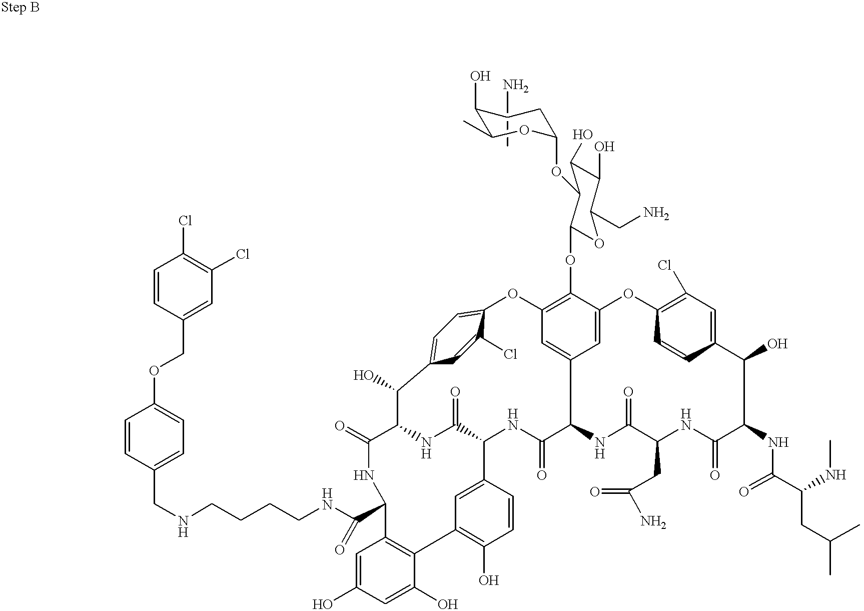

G6-amino-vancosamine TFA salt (46.0 mg, 25.7 umole), 4-(4-(3,4-dichlorobezyloxy)benzylamnio)butylamine TFA salt (47 mg, 81 umole), HOBt (28 mg, 210 umole), DIEA (28 ul, 160 umole) were dissolved in dry DMF (800ul). A solution of PyB OP (27 mg, 51 umole) in DMF (200 ul) was added dropwise. The resulting clear solution was stirred at room temperature for 45 min. The whole reaction mixture was added to ether (35 ml). The solid was collected, washed with more ether, and dried in air. The solid was dissolved in 6 ml of 1:4 CH3 CN:H20 (˜1% TFA) and purified on the preparative HPLC in three runs (10% to 35% from 0 to 1 min; 35% isocratic till 7 min; 35% to 100% from 7 to 8 min) to give, after lyophilization, 28.2 mg of product with observed m/e of 891.5 (doubly charged molecular ion) and HPLC retention time of 3.92 min.

### EXAMPLE 25

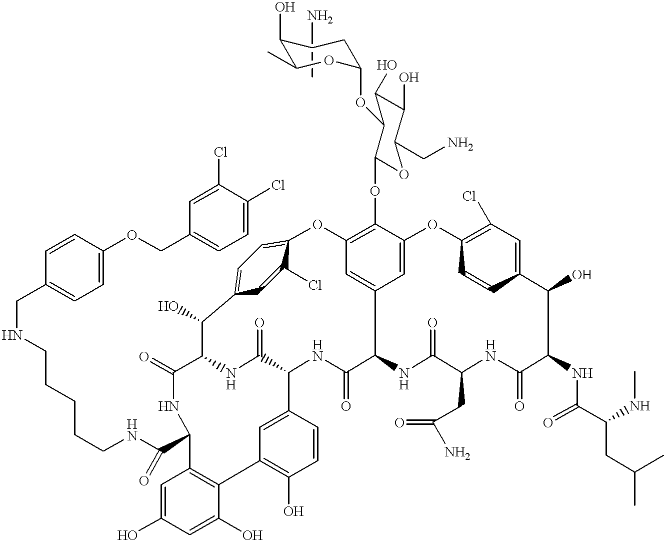

G6-amino-vancosamine TFA salt prepared in Example 24 Step A (37 mg, 21 umole), 5-(4-(3,4-dichlorobezyloxy)benzylamnio)pentylamine TFA salt (60 mg, 100 umole), HOBt (16 mg, 120 umole), DIEA (72 ul, 400 umole) were dissolved in dry DMF (500 ul) and cooled in ice/water bath. A solution of PyBOP (13.1 mg, 25 umole) in DMF (130 ul) was added dropwise. The cooling bath was removed and the clear solution was stirred for 1 hr. The reaction was monitored with HPLC. More PyBOP was added until G6-aminovancosamine is completely converted, 3 more equivalent of PyBOP was eventually used. The whole reaction mixture was added to ether (35 ml). The solid was collected, washed with more ether, and dried in air. Preparative HPLC in three runs (10% to 37% from 0 to 1 min; 37% isocratic till 7 min; 37% to 100% from 7 to 8 min) to give, after lyophilization, 8.3 mg of product with observed m/e of 898.5 (doubly charged molecular ion) and HPLC retention time of 3.94 min.

What is claimed is:

1. A compound of the formula: 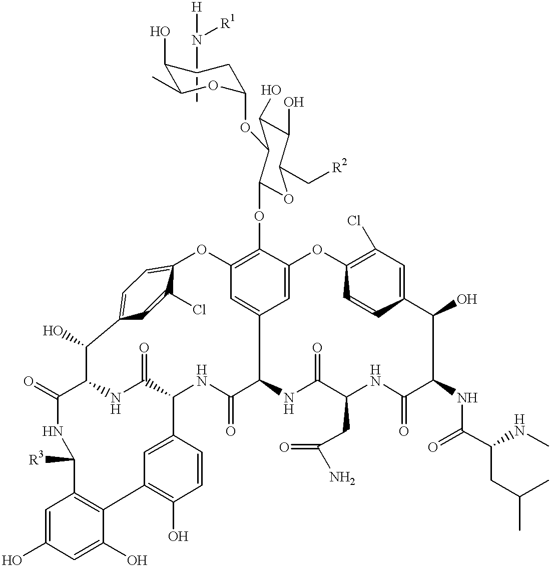
R¹ is XR^{a}; wherein X is absent or XR^{a} is — 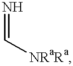
—SO₂R^{a}, —SO₂NR^{a}R^{a},—COOR^{a}, —CONR^{a}R^{a}, —COR^{a} when R^{a} is not hydrogen;
each R^{a} is independently hydrogen, alkyl, aryl, heteroaryl, substituted alkyl, substituted aryl, substituted heteroaryl; wherein
(i) each of the substituents on substituted alkyl is independently
(a) halogen,
(b) cyano,
(C) OR^{b}
(d) NR^{b}R^{c}
(e) COOR^{b}
(f) CONR^{b}R^{c},
(g) SR^{b}
(h) —SO₂R^{b}
(i) SO₂NR^{b}R^{b}
(j) aryl,
(k) aryl substituted with one or more substituents independently selected from halogen, cyano, OR^{b}, SR^{b} , COOR^{b}, CONR^{b}R^{c}, NR^{b}R^{c}, SO₂R^{b}, SO₂NR^{b}R^{c}, alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};
(l) heterocycle, or
(m) heteroaryl substituted with one or more substituents independently selected from halogen, cyano, OR^{b}, SR^{b} , COOR^{b}, CONR^{b}R^{c}, NR^{b}R^{c}, SO₂R^{b}, SO2NR^{b}R^{c}, alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};
(ii) each of the substituents on substituted aryl is independently
(a) halogen,
(b) cyano,
(C) OR^{b}
(d) NR^{b}R^{c}
(e) COOR^{b}
(f) CONR^{b}R^{c},
(g) SR^{b}
(h) SO₂R^{b}
(i) SO₂NR^{b}R^{c}
(j) aryl,
(k) aryl substituted with one or more substituents independently selected from halogen, cyano, OR^{b}, SR^{b}, COOR^{b} CONR^{b}R^{c}, NR^{b}R^{c}, SO₂R^{b}, SO2NR^{b}R^{c} alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};
(l) heterocycle, or
(m) heteroaryl substituted with one or more substituents independently selected from halogen, cyano, OR^{b}, SR^{b}, COOR^{b} CONR^{b}R^{c}, NR^{b}R^{c}, SO₂R^{b}, SO₂N^{R}bR^{c} alkyl, alkyl substituted with R^{b} , fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};
(n) alkyl,
(o) alkyl substituted with R^{b};
(p) alkenyl,
(q) alkenyl substituted with R^{b};
(r) alkynyl,
(s) alkynyl substituted with R^{b}
(iii) each of the substituents on substituted heteroaryl is independently
(a) halogen,
(b) cyano,
(C) OR^{b}
(d) NR^{b}R^{c}
(e) COOR^{b}
(f) CONR^{b}R^{c},
(g) SR^{b}
(h) SO₂R^{b}
(i) SO₂NR^{b}R^{b}
(j) aryl,
(k) aryl substituted with one or more substituents independently selected from halogen, cyano, OR^{b}, SR^{b}, COOR^{b} CONR^{b}R^{c}, NR^{b}R^{c}, SO₂R^{b}, SO2NR^{b}R^{c}, alkyl, alkyl substituted with R^{b} , fluorinated alkyl, alkenyl, alkenyl substituted with R^{b}, alkynyl, alkynyl substituted with R^{b}, aryl, aryl substituted with R^{b}, heteroaryl, heteroaryl substituted with R^{b};
(l) heterocycle, or
(m) heteroaryl substituted with one or more substituents independently selected from halogen, cyano, OR^{b}, SR^{b}, COOR^{b} CONR^{b}R^{c}, NR^{b}R^{c}, SO₂R^{b}, SO2NR^{b}R^{c} alkyl, alkyl substituted with R^{b} , fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};
(n) alkyl,
(o) alkyl substituted with R^{b};
(p) alkenyl,
(q) alkenyl substituted with R^{b};
(r) alkynyl,
(s) alkynyl substituted with R^{b};
or R^{a} and R^{a} together with the nitrogen to which they are attached form C₃-C₆ heterocycloalkyl consisting of from 2 to 5 carbons atoms and from1 or 2 nitrogen, oxygen, and sulfur atoms. The heterocycloalkyl may be substituted with alkyl, aryl, heteroaryl, OR^{b}, NR^{c}R^{b}, COOR^{b}, CONR^{b}R^{c}, substituted alkyl, substituted aryl, or substituted heteroaryl as defined above;
R^{b} and R^{c} are each independently hydrogen, alkyl, aryl, heteroaryl, substituted alkyl substituted with 1 to 3 groups of R^{x}, substituted aryl substituted with 1 to 3 groups of R^{y}, or substituted heteroaryl substituted with 1 to 3 groups of R^{z}; wherein
(i) wherein Rx represents:
(a) halogen,
(b) cyano,
(c) OH, O-alkyl
(d) N(alkyl)2, NH-alkyl
(e) OOH, COO-alkyl
(f) CON(alkyl)2, CONH-alkyl,
(g) SO2N(alkyl)2, SO2NH-alkyl,
(h) aryl,
(i) aryl substituted with one or more substituents independently selected from halogen, cyano, hydroxy, O-alkyl, alkyl, fluorinated alkyl, COOH, COO-alkyl, CONH-alkyl, CON(alkyl)2, and N(alkyl)2;
(j) heterocycle, or
(k) heterocycle substituted with one or more substituents independently selected from from halogen, cyano, hydroxy, O-alkyl, alkyl, fluorinated alkyl, COOH, COO-alkyl, CONH-alkyl, CON(alkyl)2, and N(alkyl)2;
(ii) wherein R^{y} represents:
(a) halogen,
(b) cyano,
(c) OH, O-alkyl
(d) N(alkyl)2, NH-alkyl
(e) COOH, COO-alkyl
(f) CON(alkyl)2, CONH-alkyl,
(g) SO2N(alkyl)2, SO2NH-alkyl
(h) aryl,
(i) aryl substituted with one or more substituents independently selected from halogen, cyano, hydroxy, O-alkyl, alkyl, fluorinated alkyl, COOH, COO-alkyl, CONH-alkyl, CON(alkyl)2, and N(alkyl)2;
(j) heterocycle, or
k) heterocycle substituted with one or more substituents independently selected from from halogen, cyano, hydroxy, O-alkyl, alkyl, fluorinated alkyl, COOH, COO-alkyl, CONH-alkyl, CON(alkyl)2, and N(alkyl)2;
(iii) wherein R^{z} represents:
(a) halogen,
(b) cyano,
(c) OH, O-alkyl
(d) N(alkyl)2, NH-alkyl
(e) COOH, COO-alkyl
(f) CON(alkyl)2, CONH-alkyl,
(g) SO2N(alkyl)2, SO2NH-alkyl
(h) aryl,
(i) aryl substituted with one or more substituents independently selected from halogen, cyano, hydroxy, O-alkyl, alkyl, fluorinated alkyl, COOH, COO-alkyl, CONH-alkyl, CON(alkyl)2, and N(alkyl)2;
(j) heterocycle, or
(k) heterocycle substituted with one or more substituents independently selected from from halogen, cyano, hydroxy, O-alkyl, alkyl, fluorinated alkyl, COOH, COO-alkyl, CONH-alkyl, CON(alkyl)2, and N(alkyl)2;
or Rb and Rc together with the nitrogen to which they are attached form C₃-C₆ heterocycloalkyl consisting of from 2 to 5 carbons atoms and from1 to 2 nitrogen, oxygen, and sulfur atoms, and wherein heterocycloalkyl may be substituted with alkyl, aryl, heteroaryl, O-alkyl, NHalkyl, COOH, COO-alkyl, CONH-alkyl, CON(alkyl)2, SO2N(alkyl)2, SO2NH-alkyl, substituted alkyl, substituted aryl, or substituted heteroaryl as previously defined above in R^{x}, R^{y}, R^{z}, respectively;
R2 is NR^{a}R^{a}, SR^{a}, 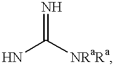
and OR^{a} wherein OR is not OH;
R³ is CONR^{a}R^{a} or COOR^{a} wherein COOR^{a} is not COOH and R^{a} does not contain as a sub-structure a generally recognized antibacterial agent.

2. The compound according to claim 1, wherein R¹ is XR^{a}; wherein X is absent and R^{a} is defined as in claim 1, R² is NR^{a}R^{a}, SR^{a}, 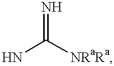
and OR^{a} is not OH;
R³ is CONR^{a}R or COOR^{a} wherein COOR^{a} is not COOH and R^{a} does not contain as a sub-structure a generally recognized antibacterial agent.

3. The compound according to claim 2, wherein
R¹ is XR^{a} wherein X is absent and each R^{a} is independently hydrogen, alkyl, and substituted alkyl; wherein
(i) each of the substituents on substituted alkyl is independently
(a) halogen,
(b) cyano,
(C) OR^{b}
(d) NR^{b}R^{c}
(e) COOR^{b}
(f) CONR^{b}R^{c},
(g) SR^{b}
(h)-SO₂R^{b}
(i) SO₂NR^{b}R^{b}
(j) aryl,
(k) aryl substituted with one or more substituents independently selected from halogen, cyano, OR^{b}, SR^{b}, COOR^{b}, CONR^{b}R^{c}, NR^{b}R^{c}, SO₂R^{b}, SO₂NR^{b}R^{c}, alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};
(1) heterocycle, or
(m) heteroaryl substituted with one or more substituents independently selected from halogen, cyano, OR^{b}, SR^{b}, COOR^{b}, CONR^{b}R^{c}, NR^{b}R^{c}, SO₂R^{b}, SO2NR^{b}R^{c}, alkyl, alkyl substituted with R^{b}, fluorinated alkyl, alkenyl, alkenyl substituted with R^{b}, alkynyl, alkynyl substituted with R^{b}, aryl, aryl substituted with R^{b}, heteroaryl, and heteroaryl substituted with R^{b};
or R^{a} and R^{a} together with the nitrogen to which they are attached form C₃-C₆ heterocycloalkyl consisting of from 2 to 5 carbons atoms and from 1 or 2 nitrogen, oxygen, and sulfur atoms; and the heterocycloalkyl may be substituted with alkyl, aryl, heteroaryl, OR^{b}, NR^{c}R^{b}, COOR^{b}, CONR^{b}R^{c}, substituted alkyl, substituted aryl, or substituted heteroaryl as defined above; and
R² and R³ are as defined in claim 2.

4. A vancomycin analog according to claim 1, wherein R¹ is unsubstituted or substituted benzyloxybenxyl wherein the substituent is one or more halogens.

5. A vancomycin analog according to claim 4, wherein the substituent is chlorine.

6. A vancomycin analog according to claim 2, wherein R¹ is 3,4-dichlorobenzyloxybenzyl.

7. A vancomycin analog according to claim 1, wherein R² is selected from the group consisting of amino, hydroxyalkylamino, phenylalkyleneamino, or a heterocyclic group.

8. A vancomycin analog according to claim 4, wherein R² is amino.

9. A vancomycin analog according to claim 4, wherein R² is hydroxyethylamino.

10. A vancomycin analog according to claim 4, wherein R² is phenethylamino.

11. A vancomycin analog according to claim 4, wherein R² is morpholino.

12. A vancomycin analog according to claim 4, wherein R² is piperidino.

13. A vancomycin analog according to claim 1, wherein R³ is selected from the group consisting of morpholinoamido, hydroxyalkoxyalkyleneamido, amino alkyleneamido and azidoalkoxyalkoxyalkoxyalkyleneamido.

14. A vancomycin analog according to claim 13, wherein R³ is morpholineamido.

15. A vancomycin analog according to claim 13, wherein R³ is hydroxyethoxyethoxyethyleneamino.

16. A vancomycin analog according to claim 13, wherein R³ is aminobutyleneamino.

17. A vancomycin analog according to claim 13, wherein R³ is azidoethoxyethoxyethoxyethyleneamino.

18. A compound according to claim 1, wherein R¹ is 3,4-dichlorobenzyloxybenzyl, R² is NH₂ and R³ is morpholinoamido.

19. A compound according to claim 1, wherein R¹ is (3,4-dichlorobenyloxy)benzyl, R² is NH₂, and R³ is hydroxyethoxyethylamido.

20. A compound according to claim 1, wherein R¹ is p-biphenylbenzyl, R² is NH₂, and R³ is morpholineamido.

21. A compound according to claim 1, wherein R¹ is p-biphenylbenzyl, R² is NH₂, and R³ is hydroxyethoxyethoxyethylamido.

22. A compound according to claim 1, wherein R¹ is p-biphenylbenzyl, R² is NH₂, and R³ is aminobutylamido.

23. A compound according to claim 1, wherein R¹ is p-biphenylbenzyl, R² is NH₂, and R³ is azidoethoxyethoxyethoxyethylamido.

24. A compound according to claim 1, wherein R¹ is (3,4-dichlorobenzyloxy)benzyl, R² is hydroxyethylamino, and R³ is morpholineamido.

25. A compound according to claim 1, wherein R¹ is (3,4-dichlorobenzyloxy)benzyl, R² is benzylamino, and R³ is morpholineamido.

26. A compound according to claim 1, wherein R¹ is (3,4-dichlorobenzyloxyl)benzyl, R is morpholino and R is morpholineamido.

27. A compound according to claim 1, wherein R¹ is (3,4-dichlorobenzyloxy)benzyl, R² is piperidino, and R³ is morpholineamido.

28. A compound according to claim 1, wherein R¹ is (3,4-dichlorobenzyloxy)benzyl, R² is (3,5-amino- ,2,4-triazoyl)mercapto, R³ is morpholineamido.

29. A compound according to claim 1, wherein R¹ is (3,4-dichlorobenzyloxy)benzyl, R² is hydroxyethylamino, and R³ is dimethylaminopropylamido.

30. A compound according to claim 1, wherein R¹ is (3,4-dichlorobenzyloxy)benzyl, R² is benzylamino, and R³ is dimethylaminopropylamido.

31. A compound according to claim 1, wherein R¹ is (3,4-dichlorobenzyloxy)benzyl, R² is morpholino, and R³ is diemthylaminopropylamido.

32. A compound according to claim 1, wherein R¹ is (3,4-dichlorobenzyloxy)benzyl, R² is piperidino, and R³ is dimethylaminopropylamido.

33. A compound according to claim 1, wherein R¹ is (3,4-dichlorobenzyloxy)benzyl, R² is (3,5-amino-1,2,4-triazoyl)mercapto, and R³ is dimethylaminopropylamido.

34. A compound according to claim 1, wherein R¹ is (3,4-dichlorobenzyloxy)benzyl, R² is NH₂, and R³ is allyloxycarbonyl.

35. A compound according to claim 1, wherein R¹ is (3,4-dichlorobenzyloxy)benzyl, R² is NH₂, and R³ is methoxycarbonyl.

36. A compound according to claim 1, wherein R¹ is (3,4-dichlorobenzyloxy)benzyl, R² is NH₂, and R³ is morpholineamido.

37. The tris trifluoroacetate salt of the compound of claim 36.

38. A compound according to claim 1, wherein R¹ is (3,4-dichlorobenzyloxy)benzyl, R² is NH₂, and R³ is dimethylaminopropylamido.

39. A compound according to claim 1, wherein R¹ is (3,4-dichlorobenzyloxy)benzyl, R² is NH₂, and R³ is aminobutyleneamido.

40. A compound according to claim 1, wherein R¹ is (3,4-dichlorobenzyloxy)benzyl, R² is NH₂, and R³ is aminoethylaminoethylamido.

41. A compound according to claim 1, wherein R¹ is NH₂, R² is NH₂, and R³ is (3,4-dichlorobenzyloxy)benzyl aminobutyleneamido.

42. A compound according to claim 1, wherein R¹ is NH₂, R² is NH₂, and R³ is (3,4-dichlorobenzyloxy)benzyl aminopentyleneamido.

43. A pharmaceutical composition comprising the analog of claim 1 or its pharmaceutically acceptable salt in combination with a pharmaceutically acceptable carrier.

44. A method of treating or preventing a bacterial infection in a mammalian patient in need thereof, comprising administering to said patient an effective amount of the analog of claim 1.
